(19) United States
(12) Patent Application Publication
(10) Pub. No.: US 2020/0360302 A1
(43) Pub. Date: Nov. 19, 2020

(54) TREATMENT OF LEIGH SYNDROME AND LEIGH-LIKE SYNDROME WITH TOCOTRIENOL QUINONES

(71) Applicant: PTC Therapeutics, Inc., Southfield, NJ (US)

(72) Inventors: Orion D. JANKOWSKI, Burlingame, CA (US); Kieron E. WESSON, Menlo Park, CA (US); Katsuyuki MURASE, Cupertino, CA (US)

(21) Appl. No.: 16/985,220

(22) Filed: Aug. 4, 2020

Related U.S. Application Data
(63) Continuation of application No. 16/213,927, filed on Dec. 7, 2018, now abandoned.
Application No. 16/213,927 is a continuation of application No. 15/288,834, filed on Oct. 7, 2016, now abandoned.
Application No. 15/288,834 is a continuation of application No. 14/094,694, filed on Dec. 2, 2013, now abandoned.
Application No. 14/094,694 is a continuation of application No. 12/982,716, filed on Dec. 30, 2010, now abandoned.
(60) Provisional application No. 61/291,784, filed on Dec. 31, 2009.

(51) Int. Cl. A61K 31/122; A61K 31/05; A61K 9/00

(52) U.S. Cl. CPC A61K 31/122; A61K 9/0053; A61K 31/05

(57) ABSTRACT

The present invention relates to methods of treating Leigh Syndrome and Leigh-like Syndrome with tocotrienol quinones, including alpha-tocotrienol quinone, in order to alleviate symptoms of the disease.

### CROSS-REFERENCE TO RELATED APPLICATIONS

This application claims priority benefit of U.S. provisional patent application No. 61/291,784, filed Dec. 31, 2009. The entire contents of that application are hereby incorporated by reference herein.

### TECHNICAL FIELD OF THE INVENTION

The present invention relates to a method of treating Leigh Syndrome and Leigh-like Syndrome with tocotrienol quinones (including tocotrienol hydroquinones), for example, alpha-tocotrienol quinone.

### BACKGROUND OF THE INVENTION

Leigh Syndrome, also known as Leigh's disease and subacute necrotizing encephalopathy, is a serious disease characterized by multiple devastating symptoms, such as psychomotor retardation, seizures, hypotonia and weakness, ataxia, eye abnormalities including vision loss, difficulty in swallowing, and lactic acidosis. The disease can result in lesions to or degeneration of the basal ganglia, thalamus, brain stem, and spinal cord. See Leigh, D., “Subacute necrotizing encephalomyelopathy in an infant,” J. Neurol. Neurosurg. Psychiat. 14:216-221 (1951). A disease termed “Leigh-like Syndrome” is also recognized, which is characterized by neurologic abnormalities atypical for but suggestive of Leigh Syndrome (Finsterer, J., “Leigh and Leigh-like syndrome in children and adults,” Pediatr. Neurol. 2008; 39:223-235). The incidence of Leigh Syndrome is estimated at 1 in 40,000 live births (Finsterer, J. ibid.) and is the most common mitochondrial disease of infancy.

Patients with Leigh Syndrome typically die before the age of five years, often from respiratory failure. Some patients with less severe disease may live to six or seven years, or even into their teen or adult years. Treatments include thiamine (Vitamin B1), Coenzyme Q, or L-carnitine and oral sodium bicarbonate or sodium citrate to manage lactic acidosis. (See the Leigh's Disease Information Page of the National Institute of Neurological Disorders and Stroke, World-Wide-Web.ninds.nih.gov/disorders/leighsdisease/leighsdisease.htm; see Finsterer, J. ibid.) Unfortunately, these treatments are not particularly effective, and the prognosis for patients with Leigh Syndrome is extremely poor. Coenzyme Q10 was used with some benefit in two sisters who survived into their late 20's and early 30's (Van Maldergem L. V. et al., Ann. Neurol. 2002; 52:750-754).

There is thus a critical and unmet need for effective treatments for Leigh Syndrome and Leigh-like Syndrome.

### SUMMARY OF THE INVENTION

In one embodiment, the invention provides methods of treating Leigh Syndrome and/or Leigh-like Syndrome with specific compounds.

In another embodiment, the invention provides methods of treating an individual suffering from Leigh Syndrome and/or Leigh-like Syndrome with tocotrienol quinones, comprising administering a therapeutically effective amount of one or more tocotrienol quinones to an individual suffering from Leigh Syndrome and/or Leigh-like Syndrome. In another embodiment, the invention provides methods of treating an individual suffering from Leigh Syndrome with alpha-tocotrienol quinone, comprising administering a therapeutically effective amount of alpha-tocotrienol quinone to an individual suffering from Leigh Syndrome. In another embodiment, the invention provides methods of treating an individual suffering from Leigh-like Syndrome with alpha-tocotrienol quinone, comprising administering a therapeutically effective amount of alpha-tocotrienol quinone to an individual suffering from Leigh-like Syndrome. In another embodiment, the invention provides methods of treating an individual suffering from Leigh Syndrome with beta-tocotrienol quinone, comprising administering a therapeutically effective amount of beta-tocotrienol quinone to an individual suffering from Leigh Syndrome. In another embodiment, the invention provides methods of treating an individual suffering from Leigh-like Syndrome with beta-tocotrienol quinone, comprising administering a therapeutically effective amount of beta-tocotrienol quinone to an individual suffering from Leigh-like Syndrome. In another embodiment, the invention provides methods of treating an individual suffering from Leigh Syndrome with gamma-tocotrienol quinone, comprising administering a therapeutically effective amount of gamma-tocotrienol quinone to an individual suffering from Leigh Syndrome. In another embodiment, the invention provides methods of treating an individual suffering from Leigh-like Syndrome with gamma-tocotrienol quinone, comprising administering a therapeutically effective amount of gamma-tocotrienol quinone to an individual suffering from Leigh-like Syndrome. In another embodiment, the invention provides methods of treating an individual suffering from Leigh Syndrome with delta-tocotrienol quinone, comprising administering a therapeutically effective amount of delta-tocotrienol quinone to an individual suffering from Leigh Syndrome. In another embodiment, the invention provides methods of treating an individual suffering from Leigh-like Syndrome with delta-tocotrienol quinone, comprising administering a therapeutically effective amount of delta-tocotrienol quinone to an individual suffering from Leigh-like Syndrome.

In another embodiment, the invention provides methods of treating an individual suffering from Leigh Syndrome and/or Leigh-like Syndrome with tocotrienol hydroquinones, comprising administering a therapeutically effective amount of one or more tocotrienol hydroquinones to an individual suffering from Leigh Syndrome and/or Leigh-like Syndrome. In another embodiment, the invention provides methods of treating an individual suffering from Leigh Syndrome with alpha-tocotrienol hydroquinone, comprising administering a therapeutically effective amount of alpha-tocotrienol hydroquinone to an individual suffering from Leigh Syndrome. In another embodiment, the invention provides methods of treating an individual suffering from Leigh-like Syndrome with alpha-tocotrienol hydroquinone, comprising administering a therapeutically effective amount of alpha-tocotrienol hydroquinone to an individual suffering from Leigh-like Syndrome. In another embodiment, the invention provides methods of treating an individual suffering from Leigh Syndrome with beta-tocotrienol hydroquinone, comprising administering a therapeutically effective amount of beta-tocotrienol hydroquinone to an individual suffering from Leigh Syndrome. In another embodiment, the invention provides methods of treating an individual suffering from Leigh-like Syndrome with beta-tocotrienol hydroquinone, comprising administering a therapeutically effective amount of beta-tocotrienol hydroquinone to an individual suffering from Leigh-like Syndrome. In another embodiment, the invention provides methods of treating an individual suffering from Leigh Syndrome with gamma-tocotrienol hydroquinone, comprising administering a therapeutically effective amount of gamma-tocotrienol hydroquinone to an individual suffering from Leigh Syndrome. In another embodiment, the invention provides methods of treating an individual suffering from Leigh-like Syndrome with gamma-tocotrienol hydroquinone, comprising administering a therapeutically effective amount of gamma-tocotrienol hydroquinone to an individual suffering from Leigh-like Syndrome. In another embodiment, the invention provides methods of treating an individual suffering from Leigh Syndrome with delta-tocotrienol hydroquinone, comprising administering a therapeutically effective amount of delta-tocotrienol hydroquinone to an individual suffering from Leigh Syndrome. In another embodiment, the invention provides methods of treating an individual suffering from Leigh-like Syndrome with delta-tocotrienol hydroquinone, comprising administering a therapeutically effective amount of delta-tocotrienol hydroquinone to an individual suffering from Leigh-like Syndrome.

In one embodiment, the pharmaceutical composition used in treating the individual comprises alpha-tocotrienol quinone, where the alpha-tocotrienol quinone comprises at least about 30% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 40% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 50% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 60% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 70% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 75% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 80% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 90% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 95% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 98% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 99% by weight of the tocotrienols and tocotrienol quinones present in the preparation.

In one embodiment, the pharmaceutical composition used in treating the individual comprises alpha-tocotrienol quinone, where the alpha-tocotrienol quinone comprises at least about 30% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 40% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 50% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 60% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 70% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 75% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 80% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 90% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 95% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 98% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 99% by weight of the material present in the preparation, excluding the weight of any added pharmaceutical carriers or excipients.

In one embodiment, the invention provides unit dosage formulations of between about 50 mg to 500 mg of alpha-tocotrienol quinone, where the purity of the alpha-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol quinones present in the preparation. The unit dosage formulations can be used to treat an individual suffering from Leigh syndrome or Leigh-like syndrome.

In one embodiment, the invention provides unit dosage formulations of between about 50 mg to 500 mg of alpha-tocotrienol quinone, where the purity of the alpha-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients. The unit dosage formulations can be used to treat an individual suffering from Leigh syndrome or Leigh-like syndrome.

Any of the embodiments of the pharmaceutical compositions, pharmaceutical formulations and unit dosage formulations of alpha-tocotrienol quinone can be used to treat an individual suffering from Leigh Syndrome or Leigh-like Syndrome, such as an individual with Leigh Syndrome, such as an individual with Leigh Syndrome where the individual has a mutation, one or more mutations, or two or more mutations in the SURF-1 gene.

In one embodiment, the pharmaceutical composition used in treating the individual comprises beta-tocotrienol quinone, where the beta-tocotrienol quinone comprises at least about 30% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 40% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 50% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 60% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 70% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 75% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 80% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 90% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 95% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 98% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 99% by weight of the tocotrienols and tocotrienol quinones present in the preparation.

In one embodiment, the pharmaceutical composition used in treating the individual comprises beta-tocotrienol quinone, where the beta-tocotrienol quinone comprises at least about 30% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 40% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 50% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 60% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 70% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 75% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 80% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 90% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 95% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 98% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 99% by weight of the material present in the preparation, excluding the weight of any added pharmaceutical carriers or excipients.

In one embodiment, the invention provides unit dosage formulations of between about 50 mg to 500 mg of beta-tocotrienol quinone, where the purity of the beta-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol quinones present in the preparation. The unit dosage formulations can be used to treat an individual suffering from Leigh syndrome or Leigh-like syndrome.

In one embodiment, the invention provides unit dosage formulations of between about 50 mg to 500 mg of beta-tocotrienol quinone, where the purity of the beta-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients. The unit dosage formulations can be used to treat an individual suffering from Leigh syndrome or Leigh-like syndrome.

Any of the embodiments of the pharmaceutical compositions, pharmaceutical formulations and unit dosage formulations of beta-tocotrienol quinone can be used to treat an individual suffering from Leigh Syndrome or Leigh-like Syndrome, such as an individual with Leigh Syndrome, such as an individual with Leigh Syndrome where the individual has a mutation, one or more mutations, or two or more mutations in the SURF-1 gene.

In one embodiment, the pharmaceutical composition used in treating the individual comprises gamma-tocotrienol quinone, where the gamma-tocotrienol quinone comprises at least about 30% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 40% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 50% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 60% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 70% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 75% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 80% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 90% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 95% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 98% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 99% by weight of the tocotrienols and tocotrienol quinones present in the preparation.

In one embodiment, the pharmaceutical composition used in treating the individual comprises gamma-tocotrienol quinone, where the gamma-tocotrienol quinone comprises at least about 30% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 40% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 50% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 60% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 70% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 75% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 80% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 90% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 95% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 98% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 99% by weight of the material present in the preparation, excluding the weight of any added pharmaceutical carriers or excipients.

In one embodiment, the invention provides unit dosage formulations of between about 50 mg to 500 mg of gamma-tocotrienol quinone, where the purity of the gamma-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol quinones present in the preparation. The unit dosage formulations can be used to treat an individual suffering from Leigh syndrome or Leigh-like syndrome.

In one embodiment, the invention provides unit dosage formulations of between about 50 mg to 500 mg of gamma-tocotrienol quinone, where the purity of the gamma-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients. The unit dosage formulations can be used to treat an individual suffering from Leigh syndrome or Leigh-like syndrome.

Any of the embodiments of the pharmaceutical compositions, pharmaceutical formulations and unit dosage formulations of gamma-tocotrienol quinone can be used to treat an individual suffering from Leigh Syndrome or Leigh-like Syndrome, such as an individual with Leigh Syndrome, such as an individual with Leigh Syndrome where the individual has a mutation, one or more mutations, or two or more mutations in the SURF-1 gene.

In one embodiment, the pharmaceutical composition used in treating the individual comprises delta-tocotrienol quinone, where the delta-tocotrienol quinone comprises at least about 30% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 40% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 50% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 60% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 70% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 75% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 80% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 90% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 95% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 98% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 99% by weight of the tocotrienols and tocotrienol quinones present in the preparation.

In one embodiment, the pharmaceutical composition used in treating the individual comprises delta-tocotrienol quinone, where the delta-tocotrienol quinone comprises at least about 30% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 40% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 50% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 60% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 70% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 75% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 80% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 90% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 95% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 98% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 99% by weight of the material present in the preparation, excluding the weight of any added pharmaceutical carriers or excipients.

In one embodiment, the invention provides unit dosage formulations of between about 50 mg to 500 mg of delta-tocotrienol quinone, where the purity of the delta-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol quinones present in the preparation. The unit dosage formulations can be used to treat an individual suffering from Leigh syndrome or Leigh-like syndrome.

In one embodiment, the invention provides unit dosage formulations of between about 50 mg to 500 mg of delta-tocotrienol quinone, where the purity of the delta-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients. The unit dosage formulations can be used to treat an individual suffering from Leigh syndrome or Leigh-like syndrome.

Any of the embodiments of the pharmaceutical compositions, pharmaceutical formulations and unit dosage formulations of delta-tocotrienol quinone can be used to treat an individual suffering from Leigh Syndrome or Leigh-like Syndrome, such as an individual with Leigh Syndrome, such as an individual with Leigh Syndrome where the individual has a mutation, one or more mutations, or two or more mutations in the SURF-1 gene.

In one embodiment, the individual suffering from Leigh Syndrome or Leigh-like Syndrome has a mutation, or at least one mutation, or two or more mutations, in a gene, or at least one gene, or two or more genes, selected from the group consisting of SURF1, MTCO3, COX10, COX15, SCO2, and TACO1. In another embodiment, the individual is suffering from Leigh Syndrome, and has a mutation, or at least one mutation, or two or more mutations, in a gene, or at least one gene, or two or more genes selected from the group consisting of SURF1, MTCO3, COX10, COX15, SCO2, and TACO1. In another embodiment, the individual suffering from Leigh Syndrome or Leigh-like Syndrome has a mutation, or has at least one mutation, or has two or more mutations, in the SURF1 gene. In another embodiment, the individual is suffering from Leigh Syndrome, and has a mutation, or has at least one mutation, or has two or more mutations, in the SURF1 gene.

In one embodiment, the individual suffering from Leigh Syndrome or Leigh-like Syndrome has a mutation, or has at least one mutation, or has two or more mutations, in a gene, or in at least one gene, or in two or more genes, said mutation(s) affecting Complex IV of the mitochondrial electron transport chain. In another embodiment, the individual is suffering from Leigh Syndrome and has a mutation, or has at least one mutation, or has two or more mutations, in a gene, or in at least one gene, or in at least two genes, said mutation(s) affecting Complex IV of the mitochondrial electron transport chain.

In one embodiment, the individual suffering from Leigh Syndrome or Leigh-like Syndrome, such as an individual suffering from Leigh Syndrome, has one or more symptoms selected from the group consisting of: one or more lesions in the central nervous system; one or more lesions in the brain; one or more lesions in the basal ganglia; one or more lesions in the thalamus; one or more lesions in the brain stem; one or more lesions in the dentate nuclei; one or more lesions in the optic nerves; one or more lesions in the spinal cord; degeneration of the central nervous system; degeneration of the brain; degeneration of the basal ganglia; degeneration of the thalamus; degeneration of the brain stem; degeneration of the dentate nuclei; degeneration of the optic nerves; degeneration of the spinal cord; progressive neurological deterioration; psychomotor retardation; mental retardation; tremors; spasms; myoclonic spasms; seizures; hypotonia; weakness; fatigue; ataxia; difficulty in walking; gastrointestinal abnormalities; eye abnormalities; vision loss; nystagmus; optic atrophy; poor reflexes; abnormal reflexes; absent reflexes; abnormal Babinski test; difficulty in breathing; difficulty in speaking; difficulty in swallowing; failure to thrive; low body weight; growth retardation; impaired kidney function; terminal stupor; lactic acidosis; poor sucking ability, loss of head control; loss of motor skills; loss of appetite; vomiting; irritability; and continuous crying.

In one embodiment, the individual suffering from Leigh Syndrome or Leigh-like Syndrome, such as an individual suffering from Leigh Syndrome, has one or more symptoms selected from the group consisting of ataxia, difficulty in walking, poor balance, inability to climb steps, inability to sit without assistance; inability to independently stand with support; inability to turn while sitting; inability to scoot or slide while sitting; inability to move extremities purposefully; inability to perform fine motor tasks; difficulty in sleeping; disrupted sleep patterns; gastrointestinal problems; impaired hand-eye coordination, and difficulty in breathing.

In one embodiment, the individual suffering from Leigh Syndrome or Leigh-like Syndrome, such as an individual suffering from Leigh Syndrome, has one or more symptoms selected from the group consisting of speech problems; difficulty in speaking in complete sentences; difficulty in enunciating; difficulty in counting aloud; poor voice and word association; cognitive difficulties, and difficulty in responding to verbal communication appropriately.

In one embodiment, administration of a therapeutically effective amount of one or more of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, or delta-tocotrienol hydroquinone, such as a therapeutically effective amount of alpha-tocotrienol quinone, to an individual suffering from Leigh Syndrome or Leigh-like Syndrome, such as an individual suffering from Leigh Syndrome, alleviates one or more symptoms selected from the group consisting of: one or more lesions in the central nervous system; one or more lesions in the brain; one or more lesions in the basal ganglia; one or more lesions in the thalamus; one or more lesions in the brain stem; one or more lesions in the dentate nuclei; one or more lesions in the optic nerves; one or more lesions in the spinal cord; degeneration of the central nervous system; degeneration of the brain; degeneration of the basal ganglia; degeneration of the thalamus; degeneration of the brain stem; degeneration of the dentate nuclei; degeneration of the optic nerves; degeneration of the spinal cord; progressive neurological deterioration; psychomotor retardation; mental retardation; tremors; spasms; myoclonic spasms; seizures; hypotonia; weakness; fatigue; ataxia; difficulty in walking; gastrointestinal abnormalities; eye abnormalities; vision loss; nystagmus; optic atrophy; poor reflexes; abnormal reflexes; absent reflexes; abnormal Babinski test; difficulty in breathing; difficulty in speaking; difficulty in swallowing; failure to thrive; low body weight; growth retardation; impaired kidney function; terminal stupor; lactic acidosis; poor sucking ability, loss of head control; loss of motor skills; loss of appetite; vomiting; irritability; and continuous crying.

In one embodiment, administration of a therapeutically effective amount of one or more of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, or delta-tocotrienol hydroquinone, such as a therapeutically effective amount of alpha-tocotrienol quinone, to an individual suffering from Leigh Syndrome or Leigh-like Syndrome, such as an individual suffering from Leigh Syndrome, alleviates one or more symptoms selected from the group consisting of: ataxia, difficulty in walking, poor balance, inability to climb steps, inability to sit without assistance; inability to independently stand with support; inability to turn while sitting; inability to scoot or slide while sitting; inability to move extremities purposefully; inability to perform fine motor tasks; difficulty in sleeping; disrupted sleep patterns; gastrointestinal problems; impaired hand-eye coordination, and difficulty in breathing.

In one embodiment, administration of a therapeutically effective amount of one or more of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, or delta-tocotrienol hydroquinone, such as a therapeutically effective amount of alpha-tocotrienol quinone, to an individual suffering from Leigh Syndrome or Leigh-like Syndrome, such as an individual suffering from Leigh Syndrome, alleviates one or more symptoms selected from the group consisting of: speech problems; difficulty in speaking in complete sentences; difficulty in enunciating; difficulty in counting aloud; poor voice and word association; cognitive difficulties, and difficulty in responding to verbal communication appropriately

In one embodiment, the compound used in treatment is able to cross the blood-brain barrier to provide a therapeutic level of compound in the central nervous system, as measured by the concentration of compound in the cerebrospinal fluid. In one embodiment, the compound used in treatment crosses the blood-brain barrier by transmembrane diffusion. In another embodiment, the compound used in treatment is administered into the cerebrospinal fluid.

In one embodiment, the compound used for treatment is administered to the patient in an amount such that the concentration of the compound in the cerebrospinal fluid of the patient is between about 0.1 ng/ml and about 10 ng/ml. In one embodiment, the compound used for treatment is administered to the patient in an amount such that the concentration of the compound in the cerebrospinal fluid of the patient is between about 0.2 ng/ml and about 5 ng/ml. In one embodiment, the compound used for treatment is administered to the patient in an amount such that the concentration of the compound in the cerebrospinal fluid of the patient is between about 0.4 ng/ml and about 3 ng/ml. In one embodiment, the compound used for treatment is administered to the patient in an amount such that the concentration of the compound in the cerebrospinal fluid of the patient is between about 0.5 ng/ml and about 2 ng/ml. In one embodiment, the compound used for treatment is administered to the patient in an amount such that the concentration of the compound in the cerebrospinal fluid of the patient is between about 0.75 ng/ml and about 2 ng/ml. In one embodiment, the compound used for treatment is administered to the patient in an amount such that the concentration of the compound in the cerebrospinal fluid of the patient is between about 1 ng/ml and about 2 ng/ml. In one embodiment, the compound used for treatment is administered to the patient in an amount such that the concentration of the compound in the cerebrospinal fluid of the patient is between about 0.75 ng/ml and about 1.5 ng/ml. In one embodiment, the compound used for treatment is administered to the patient in an amount such that the concentration of the compound in the cerebrospinal fluid of the patient is between about 1 ng/ml and about 1.5 ng/ml. In one embodiment, the compound used for treatment is administered to the patient in an amount such that the concentration of the compound in the cerebrospinal fluid of the patient is about 1.3 ng/ml.

In one embodiment, the compound used for treatment is administered to the patient in an amount such that the concentration of the compound in the cerebrospinal fluid of the patient is at or above about 0.1 ng/ml. In one embodiment, the compound used for treatment is administered to the patient in an amount such that the concentration of the compound in the cerebrospinal fluid of the patient is at or above about 0.2 ng/ml. In one embodiment, the compound used for treatment is administered to the patient in an amount such that the concentration of the compound in the cerebrospinal fluid of the patient is at or above about 0.3 ng/ml. In one embodiment, the compound used for treatment is administered to the patient in an amount such that the concentration of the compound in the cerebrospinal fluid of the patient is at or above about 0.4 ng/ml. In one embodiment, the compound used for treatment is administered to the patient in an amount such that the concentration of the compound in the cerebrospinal fluid of the patient is at or above about 0.5 ng/ml. In one embodiment, the compound used for treatment is administered to the patient in an amount such that the concentration of the compound in the cerebrospinal fluid of the patient is at or above about 0.75 ng/ml. In one embodiment, the compound used for treatment is administered to the patient in an amount such that the concentration of the compound in the cerebrospinal fluid of the patient is at or above about 1 ng/ml.

In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is between about 0.1 ng/ml and about 10 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is between about 0.2 ng/ml and about 5 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is between about 0.4 ng/ml and about 3 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is between about 0.5 ng/ml and about 2 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is between about 0.75 ng/ml and about 2 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is between about 1 ng/ml and about 2 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is between about 0.75 ng/ml and about 1.5 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is between about 1 ng/ml and about 1.5 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is about 1.3 ng/ml.

In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is at or above about 0.1 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is at or above about 0.2 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is at or above about 0.3 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is at or above about 0.4 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is at or above about 0.5 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is at or above about 0.75 ng/ml. In one embodiment, the compound used for treatment is alpha-tocotrienol quinone, and the alpha-tocotrienol quinone is administered to the patient in an amount such that the concentration of alpha-tocotrienol quinone in the cerebrospinal fluid of the patient is at or above about 1 ng/ml.

In one embodiment, the compound used for treatment is administered to the patient in an amount such that the concentration of the compound in the plasma of the patient is between about 1 ng/ml and about 5,000 ng/ml. In one embodiment, the compound used for treatment is administered to the patient in an amount such that the concentration of the compound in the plasma of the patient is between about 10 ng/ml and about 2,000 ng/ml. In one embodiment, the compound used for treatment is administered to the patient in an amount such that the concentration of the compound in the plasma of the patient is between about 10 ng/ml and about 2,000 ng/ml. In one embodiment, the compound used for treatment is administered to the patient in an amount such that the concentration of the compound in the plasma of the patient is between about 10 ng/ml and about 1,000 ng/ml. In one embodiment, the compound used for treatment is administered to the patient in an amount such that the concentration of the compound in the plasma of the patient is between about 10 ng/ml and about 500 ng/ml. In one embodiment, the compound used for treatment is administered to the patient in an amount such that the concentration of the compound in the plasma of the patient is between about 10 ng/ml and about 250 ng/ml. In one embodiment, the compound used for treatment is administered to the patient in an amount such that the concentration of the compound in the plasma of the patient is between about 10 ng/ml and about 150 ng/ml. In one embodiment, the compound used for treatment is administered to the patient in an amount such that the concentration of the compound in the plasma of the patient is between about 10 ng/ml and about 100 ng/ml. In one embodiment, the compound used for treatment is administered to the patient in an amount such that the concentration of the compound in the plasma of the patient is about 50 ng/ml.

In one embodiment, the compound used for treatment is administered to the patient in an amount such that the concentration of the compound in the plasma of the patient is at or above about 1 ng/ml. In one embodiment, the compound used for treatment is administered to the patient in an amount such that the concentration of the compound in the plasma of the patient is at or above about 5 ng/ml. In one embodiment, the compound used for treatment is administered to the patient in an amount such that the concentration of the compound in the plasma of the patient is at or above about 10 ng/ml. In one embodiment, the compound used for treatment is administered to the patient in an amount such that the concentration of the compound in the plasma of the patient is at or above about 25 ng/ml. In one embodiment, the compound used for treatment is administered to the patient in an amount such that the concentration of the compound in the plasma of the patient is at or above about 50 ng/ml. In one embodiment, the compound used for treatment is administered to the patient in an amount such that the concentration of the compound in the plasma of the patient is at or above about 75 ng/ml. In one embodiment, the compound used for treatment is administered to the patient in an amount such that the concentration of the compound in the plasma of the patient is at or above about 100 ng/ml.

In one embodiment, the compound used for treatment is alpha-tocotrienol quinone, and the alpha-tocotrienol quinone is administered to the patient in an amount such that the concentration alpha-tocotrienol quinone in the plasma of the patient is between about 1 ng/ml and about 5,000 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is between about 10 ng/ml and about 2,000 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is between about 10 ng/ml and about 2,000 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is between about 10 ng/ml and about 1,000 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is between about 10 ng/ml and about 500 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is between about 10 ng/ml and about 250 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is between about 10 ng/ml and about 150 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is between about 10 ng/ml and about 100 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is about 50 ng/ml.

In one embodiment, the compound used for treatment is alpha-tocotrienol quinone, and the alpha-tocotrienol quinone is administered to the patient in an amount such that the concentration alpha-tocotrienol quinone in the plasma of the patient is at or above about 1 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is at or above about 5 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is at or above about 10 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is at or above about 25 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is at or above about 50 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is at or above about 75 ng/ml. In one embodiment, the compound used for treatment is alpha-tocotrienol quinone, and the alpha-tocotrienol quinone is administered to the patient in an amount such that the concentration alpha-tocotrienol quinone in the plasma of the patient is at or above about 100 ng/ml.

In one embodiment, the invention embraces a method of treating an individual suffering from Leigh Syndrome or Leigh-like Syndrome, wherein the individual has a plasma lactate level greater than or equal to about 2 mmol/liter prior to treatment. In another embodiment, the invention embraces a method of treating an individual suffering from Leigh Syndrome or Leigh-like Syndrome, wherein the individual has a plasma lactate level greater than or equal to about 3 mmol/liter prior to treatment. In another embodiment, the invention embraces a method of treating an individual suffering from Leigh Syndrome or Leigh-like Syndrome, wherein the individual has a plasma lactate level greater than or equal to about 4 mmol/liter prior to treatment. The individual can be treated with alpha-tocotrienol quinone.

In one embodiment, the invention embraces a method of treating an individual suffering from Leigh Syndrome or Leigh-like Syndrome, wherein the individual has a cerebrospinal fluid lactate level greater than or equal to about 3 mmol/liter prior to treatment. In another embodiment, the invention embraces a method of treating an individual suffering from Leigh Syndrome or Leigh-like Syndrome, wherein the individual has a cerebrospinal fluid lactate level greater than or equal to about 4 mmol/liter prior to treatment. In another embodiment, the invention embraces a method of treating an individual suffering from Leigh Syndrome or Leigh-like Syndrome, wherein the individual has a cerebrospinal fluid lactate level greater than or equal to about 5 mmol/liter prior to treatment. The individual can be treated with alpha-tocotrienol quinone.

In one embodiment, the invention embraces a method of treating an individual suffering from Leigh Syndrome or Leigh-like Syndrome, wherein the individual has a plasma lactate level greater than or equal to about 2 mmol/liter and a cerebrospinal fluid lactate level greater than or equal to about 3 mmol/liter prior to treatment. In one embodiment, the invention embraces a method of treating an individual suffering from Leigh Syndrome or Leigh-like Syndrome, wherein the individual has a plasma lactate level greater than or equal to about 2 mmol/liter and a cerebrospinal fluid lactate level greater than or equal to about 4 mmol/liter prior to treatment. In one embodiment, the invention embraces a method of treating an individual suffering from Leigh Syndrome or Leigh-like Syndrome, wherein the individual has a plasma lactate level greater than or equal to about 2 mmol/liter and a cerebrospinal fluid lactate level greater than or equal to about 5 mmol/liter prior to treatment. In one embodiment, the invention embraces a method of treating an individual suffering from Leigh Syndrome or Leigh-like Syndrome, wherein the individual has a plasma lactate level greater than or equal to about 3 mmol/liter and a cerebrospinal fluid lactate level greater than or equal to about 3 mmol/liter prior to treatment. In one embodiment, the invention embraces a method of treating an individual suffering from Leigh Syndrome or Leigh-like Syndrome, wherein the individual has a plasma lactate level greater than or equal to about 3 mmol/liter and a cerebrospinal fluid lactate level greater than or equal to about 4 mmol/liter prior to treatment. In one embodiment, the invention embraces a method of treating an individual suffering from Leigh Syndrome or Leigh-like Syndrome, wherein the individual has a plasma lactate level greater than or equal to about 3 mmol/liter and a cerebrospinal fluid lactate level greater than or equal to about 5 mmol/liter prior to treatment. In one embodiment, the invention embraces a method of treating an individual suffering from Leigh Syndrome or Leigh-like Syndrome, wherein the individual has a plasma lactate level greater than or equal to about 4 mmol/liter and a cerebrospinal fluid lactate level greater than or equal to about 3 mmol/liter prior to treatment. In one embodiment, the invention embraces a method of treating an individual suffering from Leigh Syndrome or Leigh-like Syndrome, wherein the individual has a plasma lactate level greater than or equal to about 4 mmol/liter and a cerebrospinal fluid lactate level greater than or equal to about 4 mmol/liter prior to treatment. In one embodiment, the invention embraces a method of treating an individual suffering from Leigh Syndrome or Leigh-like Syndrome, wherein the individual has a plasma lactate level greater than or equal to about 4 mmol/liter and a cerebrospinal fluid lactate level greater than or equal to about 5 mmol/liter prior to treatment. The individual can be treated with alpha-tocotrienol quinone.

In one embodiment, the compound for use in treating Leigh Syndrome or Leigh-like Syndrome is selected from the group consisting of alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or any combination of two or more of the foregoing compounds, and is formulated in a pharmaceutical preparation suitable for administration via feeding tube, feeding syringe, or gastrostomy. In another embodiment, the compound for use in treating Leigh Syndrome or Leigh-like Syndrome is selected from the group consisting of alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or any combination of two or more of the foregoing compounds, and is formulated in a pharmaceutical preparation comprising one or more vegetable-derived oils, such as sesame oil, and/or one or more animal-derived oils, and/or one or more fish-derived oils. In another embodiment, the compound for use in treating Leigh Syndrome or Leigh-like Syndrome is alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or any combination of two or more of the foregoing compounds, and is formulated in a pharmaceutical preparation comprising one or more vegetable-derived oils, such as sesame oil, and/or one or more animal-derived oils, and/or one or more fish-derived oils, where the pharmaceutical preparation is suitable for administration via feeding tube, feeding syringe, or gastrostomy.

For all of the compounds and methods described herein which use a tocotrienol quinone, the quinone form can also be used in its reduced (hydroquinone, 1,4-benzenediol) form when desired. Likewise, the hydroquinone form can also be used in its oxidized (quinone) form when desired.

For all of the compounds and methods described herein, the invention also encompasses the use in treatment of the compounds and methods disclosed. The invention also encompasses the use of the compounds described herein for preparation of a medicament for use in treating Leigh Syndrome. The invention also encompasses the use of the compounds described herein for preparation of a medicament for use in treating Leigh-like Syndrome.

The present invention comprises multiple aspects, features and embodiments, where such multiple aspects, features and embodiments can be combined and permuted in any desired manner. These and other aspects, features and embodiments of the present invention will become evident upon reference to the remainder of this application, including the following detailed description. In addition, various references are set forth herein that describe in more detail certain compositions, and/or methods; all such references are incorporated herein by reference in their entirety.

### BRIEF DESCRIPTION OF THE DRAWINGS

FIG. 1 is a graph showing the viability of cells with a SURF-1 mutation from the subject treated in Example 2 in the presence of alpha-tocotrienol quinone (αTTQ, open squares) and Coenzyme Q10 (filled circles). Alpha-tocotrienol quinone displayed an EC₅₀ of 27 nM.

FIG. 2 is a graph showing the viability of cells with a SURF-1 mutation from the subject treated in Example 2 in the presence of alpha-tocotrienol quinone (αTTQ, open squares) and redox-silent alpha-tocotrienol quinone (αTTQ-RS, filled circles) (redox-silent alpha-tocotrienol quinone is 2-((6E,10E)-3-hydroxy-3,7,11,15-tetramethylhexadeca-6,10,14-trienyl)-3,5,6-trimethyl-bis(t-butyloxycarbonyl)benzene-1,4-diol). Alpha-tocotrienol quinone displayed an EC₅₀ of 21 nM.

FIG. 3 is a graph showing the oxygen consumption rate (OCR) of cells with a SURF-1 mutation from the subject treated in Example 2, in the presence of carbonylcyanide p-trifluoromethoxyphenylhydrazone (FCCP), 2-deoxyglucose (2-dG), rotenone, and Antimycin A. Filled circles: wild type. Open circles: cells with SURF-1 mutation. The agents are added sequentially; at the end of the experiment, all four agents are present in the medium.

FIG. 4 is a graph showing the Extracellular Acidification Rate (ECAR) of cells with a SURF-1 mutation from the subject treated in Example 2, in the presence of carbonylcyanide p-trifluoromethoxyphenylhydrazone (FCCP), 2-deoxyglucose (2-dG), rotenone, and Antimycin A. Open circles: wild type. Filled circles: cells with SURF-1 mutation. The agents are added sequentially; at the end of the experiment, all four agents are present in the medium.

FIG. 5 is a graph showing that alpha tocotrienol quinone crosses the blood-brain barrier; the data is from homogenized brains from C57/BL mice dosed IP at 25 mg/kg.

FIG. 6 is a graph of the dosage administered to the subject treated in Example 2 versus day of treatment.

FIG. 7 is a diagram of events observed in the subject treated in Example 2. CPK MM/MB: creatine phosphokinase (CPK) sarcomeric muscle (MM) cardiac muscle (MB); BUN/CR: blood urea nitrogen to creatinine ratio; aPTT: activated partial thromboplastin time; LFTs: Liver function tests. Q-T prolongation refers to the electrocardiogram (ECG) parameter.

FIG. 8 is a graph of plasma concentration of alpha tocotrienol quinone (αTTQ, ng/ml) in the subject treated in Example 2. Filled circles, day 1 of administration; open circles, day 14 of administration; filled squares, day 49 of administration; open squares, day 81 of administration.

FIG. 9 is a graph showing the cerebrospinal fluid (CSF) concentration of alpha tocotrienol quinone (αTTQ, ng/ml) in the subject treated in Example 2, on the 98^{th} day of treatment. The open circles are calibration samples; the filled circle is the patient sample, indicating that alpha tocotrienol quinone was present at 1.3 ng/ml in CSF.

### DETAILED DESCRIPTION OF THE INVENTION

The present invention relates to a method of treating Leigh Syndrome and/or Leigh-like Syndrome, with specific compounds.

In one aspect, tocotrienol quinones are contemplated for use in treatment, including alpha-tocotrienol quinone, beta-tocotrienol quinone, gamma-tocotrienol quinone, and delta-tocotrienol quinone. In another aspect, alpha-tocotrienol quinone is contemplated for use in treatment. Structures of tocotrienol quinones are given in Table 1 below. The tocotrienol quinones with the naturally occurring tocotrienol configuration are used in one embodiment of the invention, but other stereoisomers and/or mixtures of stereoisomers in any ratio, such as racemic mixtures, can also be used in the invention.

Tocotrienol quinones can be used in their oxidized form, as shown in Table 1, or can be used in their reduced hydroquinone form, as shown in Table 2. The quinone (cyclohexadienedione) form and hydroquinone (benzenediol) form are readily interconverted with appropriate reagents. The quinone can be treated in a biphasic mixture of an ethereal solvent with a basic aqueous solution of Na₂S₂O₄ (Vogel, A. I. et al. Vogel's Textbook of Practical Organic Chemistry, 5^{th} Edition, Prentice Hall: New York, 1996; Section 9.6.14 Quinones, “Reduction to the Hydroquinone”). Standard workup in the absence of oxygen yields the desired hydroquinone. The hydroquinone form can be oxidized to the quinone form with oxidizing agents such as ceric ammonium nitrate (CAN) or ferric chloride. The quinone and hydroquinone forms are also readily interconverted electrochemically, as is well known in the art. See, e.g., Section 33.4 of Streitweiser & Heathcock, Introduction to Organic Chemistry, New York: Macmillan, 1976.

TABLE 1
Tocotrienol quinones

| 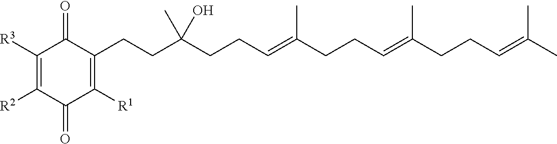 | | R¹ | R² | R³ |
|---|---|---|---|---|
| Alpha-tocotrienol quinone | 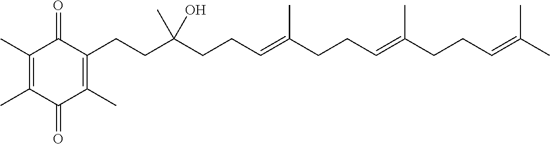 [SMILES: CC(C)=CCC/C(C)=C/CC/C(C)=C/CCC(C)(O)CCC1=C(C)C(=O)C(C)=C(C)C1=O] | methyl | methyl | methyl |
| Beta-tocotrienol quinone | 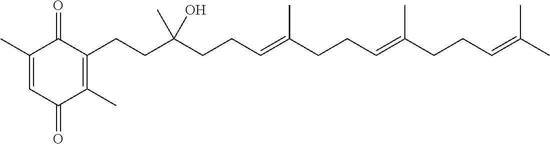 [SMILES: CC(C)=CCC/C(C)=C/CC/C(C)=C/CCC(C)(O)CCC1=C(C)C(=O)C=C(C)C1=O] | methyl | H | methyl |
| Gamma-tocotrienol quinone | 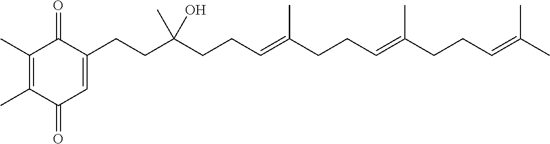 [SMILES: CC(C)=CCC/C(C)=C/CC/C(C)=C/CCC(C)(O)CCC1=CC(=O)C(C)=C(C)C1=O] | H | methyl | methyl |
| Delta-tocotrienol quinone | 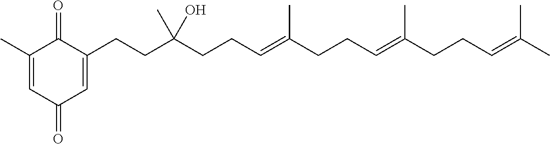 | H | H | methyl |

TABLE 2
Tocotrienol hydroquinones

| 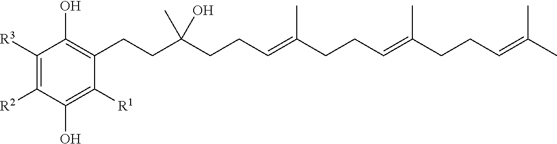 | | R¹ | R² | R³ |
|---|---|---|---|---|
| Alpha-tocotrienol hydroquinone | 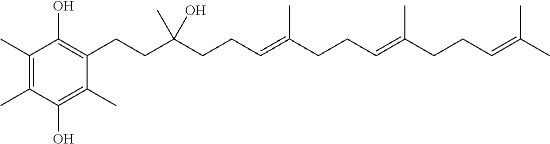 [SMILES: CC(C)=CCC/C(C)=C/CC/C(C)=C/CCC(C)(O)CCc1c(C)c(O)c(C)c(C)c1O] | methyl | methyl | methyl |
| Beta-tocotrienol hydroquinone | 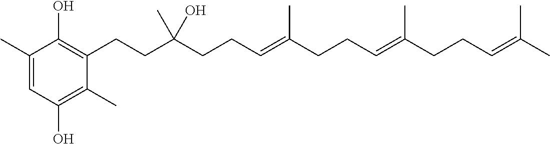 [SMILES: CC(C)=CCC/C(C)=C/CC/C(C)=C/CCC(C)(O)CCc1c(C)c(O)cc(C)c1O] | methyl | H | methyl |
| Gamma-tocotrienol hydroquinone | 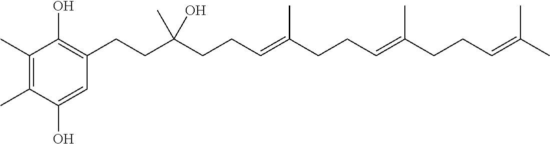 [SMILES: CC(C)=CCC/C(C)=C/CC/C(C)=C/CCC(C)(O)CCc1cc(O)c(C)c(C)c1O] | H | methyl | methyl |
| Delta-tocotrienol hydroquinone | 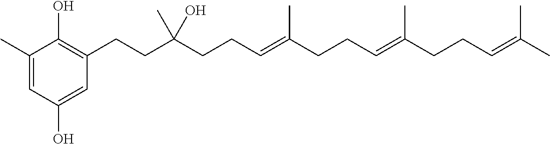 | H | H | methyl |

By “individual,” “subject,” or “patient,” is meant a mammal, preferably a human.

“Treating” a disease with the compounds and methods discussed herein is defined as administering one or more of the compounds discussed herein, with or without additional therapeutic agents, in order to reduce or eliminate either the disease or one or more symptoms of the disease, or to retard the progression of the disease or of one or more symptoms of the disease, or to reduce the severity of the disease or of one or more symptoms of the disease. “Suppression” of a disease with the compounds and methods discussed herein is defined as administering one or more of the compounds discussed herein, with or without additional therapeutic agents, in order to suppress the clinical manifestation of the disease, or to suppress the manifestation of adverse symptoms of the disease. The distinction between treatment and suppression is that treatment occurs after adverse symptoms of the disease are manifest in a subject, while suppression occurs before adverse symptoms of the disease are manifest in a subject. Suppression may be partial, substantially total, or total.

Because Leigh Syndrome and Leigh-like Syndrome are due to genetic mutations, genetic screening can be used to identify patients at risk of the disease. Leigh Syndrome and Leigh-like Syndrome can arise from mutations in Complex IV and Complex I of the mitochondrial respiratory chain. The compounds disclosed herein can be administered to, and the methods of the invention disclosed herein can be used to treat, asymptomatic patients with mutations in Complex IV and/or Complex I, who are at risk of developing the clinical symptoms of the disease, in order to suppress the appearance of any adverse symptoms or lessen the severity of symptoms that may occur. The compounds disclosed herein can be administered to, and the methods of the invention disclosed herein can be used to treat, symptomatic patients with mutations in Complex IV and/or Complex I, in order to treat the disease.

“Therapeutic use” of the compounds discussed herein is defined as using one or more of the compounds discussed herein to treat or suppress a disease, as defined above. A “therapeutically effective amount” of a compound is an amount of the compound, which, when administered to a subject, is sufficient to reduce or eliminate either a disease or one or more symptoms of a disease, or to retard the progression of a disease or of one or more symptoms of a disease, or to reduce the severity of a disease or of one or more symptoms of a disease, or to suppress the clinical manifestation of a disease, or to suppress the manifestation of adverse symptoms of a disease. A therapeutically effective amount can be given in one or more administrations.

While the compounds described herein can occur and can be used as the neutral (non-salt) compound, the description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared.

The description of compounds herein also includes all stereoisomers of the compounds, including diastereomers and enantiomers, and mixtures of stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

The compounds can be administered in prodrug form. Prodrugs are derivatives of the compounds, which are themselves relatively inactive but which convert into the active compound when introduced into the subject in which they are used by a chemical or biological process in vivo, such as an enzymatic conversion. Suitable prodrug formulations include, but are not limited to, peptide conjugates of the compounds disclosed herein and esters of compounds disclosed herein. Further discussion of suitable prodrugs is provided in H. Bundgaard, Design of Prodrugs, New York: Elsevier, 1985; in R. Silverman, The Organic Chemistry of Drug Design and Drug Action, Boston: Elsevier, 2004; in R. L. Juliano (ed.), Biological Approaches to the Controlled Delivery of Drugs (Annals of the New York Academy of Sciences, v. 507), New York: New York Academy of Sciences, 1987; and in E. B. Roche (ed.), Design of Biopharmaceutical Properties Through Prodrugs and Analogs (Symposium sponsored by Medicinal Chemistry Section, APhA Academy of Pharmaceutical Sciences, November 1976 national meeting, Orlando, Fla.), Washington: The Academy, 1977.

### Monitoring Treatment Efficacy Using Biomarkers

Several metabolic biomarkers can be used to monitor the efficacy of compounds in treatment of Leigh Syndrome and Leigh-like Syndrome. These biomarkers include, but are not limited to, lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H⁺) or NADPH (NADPH+H⁺) levels; NAD or NADP levels; ATP levels; anaerobic threshold; reduced coenzyme Q (CoQ^{red}) levels; oxidized coenzyme Q (CoQ^{ox}) levels; total coenzyme Q (CoQ^{tot}) levels; oxidized cytochrome c levels; reduced cytochrome c levels; oxidized cytochrome c/reduced cytochrome c ratio; acetoacetate levels, β-hydroxy butyrate levels, acetoacetate/β-hydroxy butyrate ratio, 8-hydroxy-2′-deoxyguanosine (8-OHdG) levels; levels of reactive oxygen species; and levels of oxygen consumption (VO2), levels of carbon dioxide output (VCO2), and respiratory quotient (VCO2/VO2). Exercise intolerance can also be used as an indicator of the efficacy of compounds in treatment of Leigh Syndrome and Leigh-like Syndrome. Several of these clinical markers are measured routinely in exercise physiology laboratories, and provide convenient assessments of the metabolic state of a subject.

Pyruvate, a product of the metabolism of glucose, is removed by reduction to lactic acid in an anaerobic setting; the degree to which this process occurs is dependent on the function of the mitochondrial respiratory chain. Dysfunction of the respiratory chain can lead to an abnormally high conversion of pyruvate to lactate, supported by the elevated lactate/pyruvate ratios observed in mitochondrial cytopathies (Scriver CR. The metabolic and molecular bases of inherited disease. 7th ed. New York: McGraw-Hill, Health Professions Division; 1995; Munnich A, Rustin P, Rotig A, et al. Clinical aspects of mitochondrial disorders. J Inherit Metab Dis. 1992; 15(4):448-455). Blood lactate/pyruvate ratio is, therefore, widely used as a noninvasive test for detection of mitochondrial cytopathies and toxic mitochondrial myopathies. (See Chariot P, Ratiney R, Ammi-Said M, Herigault R, Adnot S, Gherardi R. Optimal handling of blood samples for routine measurement of lactate and pyruvate. Arch Pathol Lab Med. July 1994; 118(7):695-697; Chariot P, Monnet I, Mouchet M, et al. Determination of the blood lactate:pyruvate ratio as a noninvasive test for the diagnosis of zidovudine myopathy. Arthritis Rheum. April 1994; 37(4):583-586.) Total concentration levels of lactate and total concentration levels of pyruvate that are elevated above the normal range are also observed in Leigh Syndrome, and those elevated total concentrations can serve as additional biomarkers in addition to the elevated lactate/pyruvate ratio. Another biomarker which can be monitored is CoQ₁₀ concentration.

Biomarkers and techniques for measurement of biomarkers that can be used to monitor the efficacy of treatment include, but are not limited to:

Magnetic Resonance Spectroscopy:

Brain lactate measurement and quantification directly reflect cellular electron balance and indirectly reflect energy production. Magnetic resonance spectroscopy can be used to assess metabolic parameters of the brain with a focus on lactate, i.e., central nervous system (CNS) concentration of lactate and lactate/pyruvate ratio. MRS has been used to measure lactate using proton MRS (1H-MRS) (Kaufmann et al., Neurology 62(8):1297-302 (2004)). Phosphorous MRS (31P-MRS) has been used to demonstrate low levels of cortical phosphocreatine (PCr) (Matthews et al., Ann. Neurol. 29(4):435-8 (1991)), and a delay in PCr recovery kinetics following exercise in skeletal muscle (Matthews et al., Ann. Neurol. 29(4):435-8 (1991); Barbiroli et al., J. Neurol. 242(7):472-7 (1995); Fabrizi et al., J. Neurol. Sci. 137(1):20-7 (1996)).

Proton Magnetic Resonance Spectroscopy (MRS) is used to measure the levels of different metabolic compounds in the brain of mitochondrial patients, that emit a unique resonance frequency expressed as chemical shifts in parts per million (ppm). Patients with mitochondrial disease are evaluated for lactate, N-acetyl aspartate (NAA), succinate, total creatine, choline (Cho) and myo-inositol. Lactate is not detected in normal patients; however, if metabolism shifts to anaerobic glycolysis in mitochondrial respiratory chain deficiencies, lactate levels increase (see Barkovich and et al, AJNR Am. J. Neuroradiol. (1993) 14, (5) 1119-1137). One of the best biomarkers for neuronal integrity is NAA which is localized to neurons and dendrites (see Clark, J B, Dev. Neurosci. (1998), 20 (4-5)271-276). Reductions of NAA levels when normalized to creatine are seen in mitochondrial disease patients. The signal for choline (Cho) includes free choline, phosphoryl choline and phosphatidylcholine which constitute myelin. Cho elevations reflect membrane turnover and demyelination. Deficient respiratory chain activity produces increases in succinate concentration detectable by this method (see Brockmann et al., Ann. Neurol. (2002)52 (1) 38-45). Proton MRS obtained over conventional MRI, provides additional information through visualization of metabolic changes.

Lactic Acid (Lactate) Levels:

Brain lactate measurement and quantification directly reflect cellular electron balance and indirectly reflect energy production. Lactate levels can be measured by taking samples of appropriate bodily fluids such as whole blood, plasma, or cerebrospinal fluid. Using magnetic resonance, lactate levels can be measured in virtually any volume of the body desired, such as the brain. Whole blood, plasma, and cerebrospinal fluid lactate levels can be measured by commercially available equipment such as the YSI 2300 STAT Plus Glucose & Lactate Analyzer (YSI Life Sciences, Ohio).

NAD, NADP, NADH and NADPH Levels:

Measurement of NAD, NADP, NADH (NADH+H⁺) or NADPH (NADPH+H⁺) can be measured by a variety of fluorescent, enzymatic, or electrochemical techniques, e.g., the electrochemical assay described in US 2005/0067303.

Oxygen consumption (vO₂ or VO2), carbon dioxide output (vCO₂ or VCO2), and respiratory quotient (RQ=VCO2/VO2):

vO₂ is usually measured either while resting (resting vO₂) or at maximal exercise intensity (vO₂ max). Optimally, both values will be measured. However, for severely disabled patients, measurement of vO₂ max may be impractical. Measurement of both forms of vO₂ is readily accomplished using standard equipment from a variety of vendors, e.g., Korr Medical Technologies, Inc. (Salt Lake City, Utah). VCO2 can also be readily measured, and the ratio of VCO2 to VO2 under the same conditions (VCO2/VO2, either resting or at maximal exercise intensity) provides the respiratory quotient (RQ).

Other Oxygen Metabolism Deficiencies:

Other problems with oxygen metabolism which can be measured include deficit in peripheral oxygen extraction (A-VO2 difference) and an enhanced oxygen delivery (hyperkinetic circulation) (Taivassalo et al., Brain 126(Pt 2):413-23 (2003)). This can be demonstrated by a lack of exercise induced deoxygenation of venous blood with direct AV balance measurements (Taivassalo et al., Ann. Neurol. 51(1):38-44 (2002)) and non-invasively by near infrared spectroscopy (Lynch et al., Muscle Nerve 25(5):664-73 (2002); van Beekvelt et al., Ann. Neurol. 46(4):667-70 (1999)). Decreased affinity for oxygen of cytochrome-c oxidase has been observed in cultured fibroblasts from Leigh syndrome patients with SURF1 mutations (Pecina et al., Am. J. Physiol. Cell Physiol. 287(5):C1384-8 (2004)).

Oxidized Cytochrome c, Reduced Cytochrome c, and Ratio of Oxidized Cytochrome c to Reduced Cytochrome c:

Cytochrome c parameters, such as oxidized cytochrome c levels (Cyt Cₒₓ), reduced cytochrome c levels (Cyt C_{red}), and the ratio of oxidized cytochrome c/reduced cytochrome c ratio (Cyt Cₒₓ)/(Cyt C_{red}), can be measured by in vivo near infrared spectroscopy. See, e.g., Rolfe, P., “In vivo near-infrared spectroscopy,” Ann. Rev. Biomed. Eng. 2:715-54 (2000) and Strangman et al., “Non-invasive neuroimaging using near-infrared light” Biol. Psychiatry 52:679-93 (2002). See also Pecina et al., Am. J. Physiol. Cell Physiol. 287(5):C1384-8 (2004), showing decreased affinity for oxygen of cytochrome-c oxidase in cultured fibroblasts from Leigh syndrome patients with SURF1 mutations.

Exercise Tolerance/Exercise Intolerance:

Exercise intolerance is defined as “the reduced ability to perform activities that involve dynamic movement of large skeletal muscles because of symptoms of dyspnea or fatigue” (Piña et al., Circulation 107:1210 (2003)). Exercise intolerance is often accompanied by myoglobinuria, due to breakdown of muscle tissue and subsequent excretion of muscle myoglobin in the urine. Various measures of exercise intolerance can be used, such as time spent walking or running on a treadmill before exhaustion, time spent on an exercise bicycle (stationary bicycle) before exhaustion, and similar tests.

Acetoacetate/3-Hydroxybutyrate (Acetoacetate/β-Hydroxybutyrate) Ratio:

Changes in the redox state of liver mitochondria can be investigated by measuring the arterial ketone body ratio (acetoacetate/3-hydroxybutyrate: AKBR) (Ueda et al., J. Cardiol. 29(2):95-102 (1997)).

8-hydroxy-2′-deoxyguanosine (8-OHdG):

Urinary excretion of 8-hydroxy-2′-deoxyguanosine (8-OHdG) often has been used as a biomarker to assess the extent of repair of ROS-induced DNA damage in both clinical and occupational settings (Erhola et al., FEBS Lett. 409(2):287-91 (1997); Honda et al., Leuk. Res. 24(6):461-8 (2000); Pilger et al., Free Radic. Res. 35(3):273-80 (2001); Kim et al. Environ Health Perspect 112(6):666-71 (2004)).

Routine Plasma Analytes:

Blood ketone body ratios, including lactate: pyruvate and beta-hydroxy butyrate:acetoacetate, reflect electron balance. Alterations in these ratios can be used to assess systemic metabolic function. Increased blood lactate, increased blood pyruvate, increased blood alanine, and blood pH (to check for metabolic acidosis) can also be monitored.

Other Blood, Metabolic, or Enzymatic Biomarkers:

patients can be monitored for an increase in the number of white blood cells, and for cytochrome c oxidase deficiency.

Routine Measures of Cardiac Function:

Mitochondrial diseases are frequently characterized by altered heart function. 12-lead ECG can be employed to measure QT/QTc. Transthoracic echocardiography can be used to assess dynamic cardiac function.

Measurements of Brainstem Function:

brainstem auditory evoked response (BAER), somatosensory-evoked potentials (SEP or SSEP), blink reflex, and polysomnography (PSG) can be monitored in patients to assess brainstem function.

Other Reflexes:

Babinski test (Babinski reflex, Babinski sign), which can indicate motor neuron damage.

Metabolomic Analysis of Plasma and Urine:

Urine analysis can be performed on the patient, and can include measurement of the following organic acids: lactic acid, pyruvic acid, succinic acid, fumaric acid, 2-ketoglutaric acid, methyl malonic acid, 3-OH butyric acid, acetoacetic acid, 2-keto-3-methylvaleric acid, 2-keto-isocaproic acid, 2-keto-isovaleric acid, ethylmalonic acid, adipic acid, suberic acid, sebacic acid, 4-OH-phenylacetic acid, 4-OH-phenyllactic acid, 4-OH-phenylpyruvic acid, succinylacetone, and creatinine. Urine analysis performed on the patient can also include measurement of the following amino acids: proline, glutamine, threonine, serine, glutamic acid, arginine, glycine, alanine, histidine, lysine, valine, asparagine, methionine, phenylalanine, isoleucine, leucine, tyrosine, hydroxyproline, creatinine, aspartic acid, cysteine, ornithine, citrulline, homocysteine, and taurine. In a panel of metabolic analytes, the following can be measured: sodium, potassium, chloride, bicarbonate, anion gap, glucose (serum), urea nitrogen (blood), creatinine, calcium, bilirubin, aspartate amino transferase, alanine amino transferase, alkaline phosphatase, total protein (serum), albumin (serum), and hemolysis index. Recently, the Critical Path Initiative has put forth a battery of biomarkers to predict drug toxicity that can also reflect renal mitochondrial function. Alterations in KIM-1, Albumin, Total Protein, β2-microglobulin, Cystatin C, Clusterin, Trefoil Factor-3, and Neutrophil Gelatinase—Associated Lipocalin can be used to both detect (if present) a subclinical nephropathy and assemble a more accurate depiction of the natural history of SURF1 renal function. Finally, Haas, et al. Mol Genet Metab. (2008) 94(1):16-37 describes various tests, such as MRS-based biochemical analysis, that can be used in the present invention.

### Leigh Syndrome and Leigh-Like Syndrome: Symptoms Amenable to Treatment

Leigh Syndrome gives rise to several devastating symptoms, including lesions in, or degeneration of, the brain and central nervous system, including basal ganglia, thalamus, brain stem, dentate nuclei, optic nerves, and spinal cord; progressive neurological deterioration; psychomotor retardation; mental retardation; tremors; spasms, including myoclonic spasms; seizures; hypotonia and/or weakness; fatigue; ataxia and/or difficulty in walking; gastrointestinal abnormalities; eye abnormalities including vision loss, nystagmus, and/or optic atrophy; hearing loss; poor, abnormal, or absent reflexes, including abnormal Babinski test; difficulty in breathing; difficulty in speaking; difficulty in swallowing; failure to thrive; low body weight; growth retardation; impaired kidney function; terminal stupor; and lactic acidosis. In infants, Leigh Syndrome is characterized by poor sucking ability, loss of head control, loss of motor skills, loss of appetite, vomiting, irritability, continuous crying, and seizures.

Symptoms of Leigh-like Syndrome are similar to those of Leigh Syndrome, although they may not be as severe, and also include symptoms atypical of Leigh Syndrome. These atypical symptoms include peripheral nervous system pathology such as polyneuropathy or myopathy, and non-neurologic pathology such as diabetes, short stature, excessive growth of hair (hypertrichosis), cardiomyopathy, anemia, renal failure, vomiting, or diarrhea (see Finsterer, J., “Leigh and Leigh-like syndrome in children and adults,” Pediatr. Neurol. 2008; 39:223-235).

In one embodiment, the methods of the invention can alleviate one or more symptoms of Leigh Syndrome or Leigh-like Syndrome, including one or more lesions in the central nervous system; one or more lesions in the brain; one or more lesions in the basal ganglia; one or more lesions in the thalamus; one or more lesions in the brain stem; one or more lesions in the dentate nuclei; one or more lesions in the optic nerves; one or more lesions in the spinal cord; degeneration of the central nervous system; degeneration of the brain; degeneration of the basal ganglia; degeneration of the thalamus; degeneration of the brain stem; degeneration of the dentate nuclei; degeneration of the optic nerves; degeneration of the spinal cord; progressive neurological deterioration; demyelination; sensory neuropathy; psychomotor retardation; mental retardation; tremors; spasms, including myoclonic spasms; seizures; hypotonia and/or muscle weakness; fatigue; ataxia and/or difficulty in walking; gastrointestinal abnormalities; eye abnormalities including vision loss, nystagmus, optic atrophy and/or pigmentary retinopathy; hearing loss; poor, abnormal, or absent reflexes, including abnormal Babinski test; difficulty in breathing; difficulty in speaking; difficulty in forming words; difficulty in swallowing; failure to thrive; low body weight; growth retardation; impaired kidney function; terminal stupor; and lactic acidosis. In infants, Leigh Syndrome is characterized by poor sucking ability, loss of head control, loss of motor skills, loss of appetite, vomiting, irritability, continuous crying, and seizures. In another embodiment, the methods of the invention can alleviate one or more symptoms of Leigh Syndrome or Leigh-like Syndrome, including one or more lesions in the central nervous system, one or more lesions in the brain, one or more lesions in the basal ganglia, one or more lesions in the thalamus, one or more lesions in the brain stem, one or more lesions in the dentate nuclei, one or more lesions in the optic nerves, one or more lesions in the spinal cord, degeneration of the central nervous system, degeneration of the brain, degeneration of the basal ganglia, degeneration of the thalamus, degeneration of the brain stem, degeneration of the dentate nuclei, degeneration of the optic nerves, and degeneration of the spinal cord.

In one embodiment, the methods of the invention can alleviate one or more symptoms of Leigh Syndrome or Leigh-like Syndrome, including failure to thrive, swallowing dysfunction, optic atrophy, inability to speak, inability to walk, gastrointestinal problems, tremors, or abnormal Babinski test.

In another embodiment, treatment according to the invention can produce in a patient an adequate reduction or alleviation of one or more of the observable characteristics of Leigh Syndrome by an amount that is discernible to a human observer, such as a parent, physician or caretaker, without the use of special devices such as imaging technology, microscopes or chemical analytical devices. For example, treatment according to the invention can produce an observable reduction of ataxia and difficulty in walking, wherein a patient that was bed-bound and lethargic prior to treatment is able, after treatment, to walk with assistance; balance, including balancing on one foot; ride a tricycle; walk up steps; sit without assistance; independently stand and support himself or herself by holding on to a table or a fixed object for at least one minute; turn and scoot or slide while sitting; move his or her extremities purposefully, as in giving a “high-five” gesture; and perform fine motor tasks such as grasping small objects. Treatment according to the invention can produce an observable reduction of speech problems, such as speaking in complete sentences, improved enunciation, counting aloud, having increased voice and word association; and can improve cognitive skills, such as asking “why,” and responding to verbal communication appropriately. Treatment according to the invention can produce observable improved sleep patterns, normalization of gastrointestinal problems, improved hand-eye coordination, and improved breathing.

Standard motor function tests can be used to assess many of these symptoms, including tests used by physical therapists, occupational therapists, and rehabilitation medicine specialists to assess patient function. As many patients presenting with Leigh Syndrome or Leigh-like Syndrome are young (five to six years old or younger), age-appropriate tests are used.

There are several known assessment products for pediatricians to evaluate children. For physical abilities, the Pediatric Evaluation of Disability Inventory (PEDI) can be used (see Haley, S. M., Coster, W. J., Ludlow, L. H., Haltiwanger, J. T., & Andrellos, P. J. (1992). Pediatric Evaluation of Disability Inventory: Development, Standardization, and Administration Manual, Version 1.0. Boston, Mass.: Trustees of Boston University, Health and Disability Research Institute); PEDI enables evaluation of functional disabilities using standardized score forms. The PEDI can be used to assess key functional capabilities and performance in children ages six months to seven years, and to evaluate older children whose functional abilities are lower than those of seven-year-olds without disabilities. PEDI can be used to identify functional deficits and monitor treatment progress.

For neuro-psychiatric evaluation, the NEPSY-II assessment (Korkman, Marit; Kirk, Ursula; & Kemp, Sally. (2007) NEPSY-II—Second Edition, San Antonio, Tex.: Pearson) can be used to gauge neuropsychological development. Testing in children 3-4 years of age can assess six functional domains: attention and executive functions; language and communication; sensorimotor functions; visuospatial functions; learning and memory; and social perception.

Additionally Wolf N. I. et al., “Mitochondrial disorders: a proposal for consensus diagnostic criteria in infants and children,” Neurology (2002) 59 (9) 1402-1405 also describes diagnostic criteria in infants and children with mitochondrial diseases.

A scale to monitor progression and treatment of mitochondrial diseases in children, commonly known as the Newcastle Paediatric Mitochondrial Disease Scale (NPMDS), monitors the biophysical markers of disease progression. The scale is based around four domains: current function; system-specific involvement; current clinical assessment; and quality of life, as described by C. Phoenix et al, “A scale to monitor progression and treatment of mitochondrial disease in children,” Neuromuscular Disorders (2006) 16 814-820.

### Mutations Causing Leigh Syndrome

Several mutations in genes involved in energy metabolism are implicated in Leigh Syndrome. Mutations identified occur in both nuclear-encoded genes and mitochondrial-encoded genes. Most of the mutations affect the mitochondrial electron transport chain.

Individuals with mutations in these genes who do not presently manifest symptoms of Leigh Syndrome or Leigh-like Syndrome, can be treated with the methods of the invention in order to suppress symptoms of Leigh Syndrome or Leigh-like Syndrome, or to lessen the severity of symptoms of Leigh Syndrome or Leigh-like Syndrome once they develop. Accordingly, in one aspect, the invention comprises methods of administering specific compounds, such as tocotrienol quinones, to individuals who have one or more of the mutations listed herein. In another aspect, the invention comprises methods of administering alpha-tocotrienol quinone to individuals who have one or more of the mutations listed herein.

Leigh Syndrome and Leigh-like Syndrome arising from mutations that affect Complex W are of interest for the present invention. These mutations include mitochondrial-encoded MTCO3; nuclear-encoded COX10, COX15, SCO2, SURF1, which is involved in the assembly of complex IV, and TACO1. These mutations are discussed at World-Wide-Web.ncbi.nlm.nih.gov/entrez/dispomim.cgi?id=256000.

A gene of interest in the present invention is the SURF1 gene. SURF1 is a nuclear-encoded gene that codes for a cytochrome C oxidase (Complex IV) assembly protein. Consequently, mutations in SURF1 result in respiratory chain diseases. In one aspect, the invention embraces treatment of patients with Leigh Syndrome or Leigh-like Syndrome having a mutation, or having at least one mutation, or having two or more mutations, in the SURF1 gene.

### Dosages

The compounds used in the methods of the invention can be administered in various amounts. Examples of daily dosages which can be used are an effective amount within the dosage range of about 0.1 mg/kg to about 300 mg/kg body weight, or within about 0.1 mg/kg to about 100 mg/kg body weight, or within about 0.1 mg/kg to about 80 mg/kg body weight, or within about 0.1 mg/kg to about 50 mg/kg body weight, or within about 0.1 mg/kg to about 30 mg/kg body weight, or within about 0.1 mg/kg to about 10 mg/kg body weight, or within about 1.0 mg/kg to about 80 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 80 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight, or about 0.1, about 5, about 10, about 15, about 20, about 25, about 30, about 40, about 50, about 60, about 70, about 75, about 80, about 90, about 100, about 125, about 150, about 175, about 200, about 225, about 250, about 275, about 300, about 325, about 350, about 375, about 400, about 425, about 450, about 500, about 550, about 600, about 650, about 700, about 750, about 800, about 850, about 900, about 950, or about 1000 mg total. The compound(s) may be administered in a single daily dose, or the total daily dosage may be administered in divided dosages of two, three or four times daily. These dosages can be administered long term, for example, over months, years, or even over the entire lifetime of the patient.

The particular dosage appropriate for a specific patient is determined by dose titration. For example, animal studies of alpha-tocotrienol quinone administration have shown that in rats, at 10 mg/kg, bioavailability is high (˜90%), Cₘₐₓ=931 ng/mL, Tₘₐₓ=3.5 h and t_{1/2}=3.5 h. There is less than dose-proportionality since for an increase in doses of 2.4:6:10:20 there is only an increase in AUCs of 1.5:2.8:4.0:6.7. This lack of dose-proportionality may be due to decreased absorption since there is no change in t_{1/2} over dose range. Alpha-tocotrienol quinone tested in rats was safe when given acutely up to 2000 mg/kg. In fasted dogs, at 10 mg/kg, bioavailability is low (˜16%), Cₘₐₓ=442 ng/mL, Tₘₐₓ=2.8 h and t_{1/2}=7.6 h.

The single dose and repeat dose plasma profiles for alpha tocotrienol quinone were simulated using a dose adjusted to achieve a Cₘₐₓ<10 μM and a Cₘᵢₙ>0.5 μM. Assuming a daily dose and linear kinetics, for a 70 kg adult the total dose would need to be 379 mg (5.41 mg/kg) to achieve a C₂₄ₕ of 220.5 ng/ml (0.5 μM). The dose is adjusted as appropriate, as many patients with Leigh Syndrome or Leigh-like Syndrome are children weighing much less than 70 kg.

The starting dose can be estimated based on the United States Food and Drug Administration guidelines titled “Estimating the Maximum Safe Starting Dose in Initial Clinical Trials for Therapeutics in Adult Healthy Volunteers” (July 2005) as well as the International Conference on Harmonisation of Technical Requirements for Registration of Pharmaceuticals for Human Use (ICH) guidelines titled “Guidance on Non-clinical Safety Studies for the Conduct of Human Clinical Trials and Marketing Authorization for Pharmaceuticals” (July 2008). Per ICH guidelines, predicted exposures from the starting dose should not exceed 1/50^{th} the NOAEL (No-Adverse-Observed-Effect-Level) in the more sensitive species on a mg/m² basis. Following a single oral dose of alpha-tocotrienol quinone, the NOAEL was established to be 500 mg/kg for the female rat, i.e. 3,000 mg/m2. This dosage would be equivalent to 81 mg/kg in an adult human. 1/50th of 81 mg/kg is 1.6 mg/kg, i.e. 110 mg for a 70 kg adult, or 16 mg for a 10 kg child. This dose can be administered once, twice, or three times daily.

### Co-Administered Agents

While the compounds described herein can be administered as the sole active pharmaceutical agent, they can also be used in combination with one or more other agents used in the treatment or suppression of Leigh Syndrome or Leigh-like Syndrome. Representative agents useful in combination with the compounds described herein for the treatment or suppression of Leigh Syndrome or Leigh-like Syndrome include, but are not limited to, Coenzyme Q, including Coenzyme Q10; idebenone; MitoQ; acetylcarnitine (such as acetyl-L-carnitine or acetyl-DL-carnitine); palmitoylcarnitine (such as palmitoyl-L-carnitine or palmitoyl-DL-carnitine); carnitine (such as L-carnitine or DL-carnitine); quercetine; mangosteen; acai; uridine; N-acetyl cysteine (NAC); polyphenols, such as resveratrol; Vitamin A; Vitamin C; lutein; beta-carotene; lycopene; glutathione; fatty acids, including omega-3 fatty acids such as α-linolenic acid (ALA), eicosapentaenoic acid (EPA), and docosahexaenoic acid (DHA); lipoic acid and lipoic acid derivatives; Vitamin B complex; Vitamin B1 (thiamine); Vitamin B2 (riboflavin); Vitamin B3 (niacin, nicotinamide, or niacinamide); Vitamin B5 (pantothenic acid); Vitamin B6 (pyridoxine or pyridoxamine); Vitamin B7 (biotin); Vitamin B9 (folic acid, also known as Vitamin B11 or Vitamin M); Vitamin B12 (cobalamins, such as cyanocobalamin); inositol; 4-aminobenzoic acid; folinic acid; Vitamin E; other vitamins; and antioxidant compounds.

The co-administered agents can be administered simultaneously with, prior to, or after, administration of the primary compound intended to treat Leigh Syndrome or Leigh-like Syndrome.

### Formulations and Routes of Administration

The compounds used in the methods of the invention may be administered in any suitable form that will provide sufficient plasma and/or central nervous system levels of the compounds. The compounds can be administered enterally, orally, parenterally, sublingually, by inhalation (e.g. as mists or sprays), rectally, or topically in unit dosage formulations containing conventional nontoxic pharmaceutically acceptable carriers, excipients, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous injection, intraarterial injection, intramuscular injection, intrasternal injection, or infusion techniques. The compounds are mixed with pharmaceutically acceptable carriers, excipients, adjuvants, and vehicles appropriate for the desired route of administration.

In certain embodiments of the invention, especially those embodiments where a formulation is used for injection or other parenteral administration including the routes listed herein, but also including embodiments used for oral, gastric, gastrointestinal, or enteric administration, the formulations and preparations used in the methods of the invention are sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

Oral administration is advantageous due to its ease of implementation and patient (or caretaker) compliance. However, patients with Leigh Syndrome or Leigh-like Syndrome often have difficulty in swallowing. Introduction of medicine via feeding tube, feeding syringe, or gastrostomy can be employed in order to accomplish enteric administration. The active compound (and, if present, other co-administered agents) can be enterally administered in sesame oil, or any other pharmaceutically acceptable carrier suitable for formulation for administration via feeding tube, feeding syringe, or gastrostomy.

The term “nutraceutical” has been used to refer to any substance that is a food or a part of a food and provides medical or health benefits, including the prevention and treatment of disease. Hence, compositions falling under the label “nutraceutical” may range from isolated nutrients, dietary supplements and specific diets to genetically engineered designer foods, herbal products, and processed foods such as cereals, soups and beverages. In a more technical sense, the term has been used to refer to a product isolated or purified from foods, and generally sold in medicinal forms not usually associated with food and demonstrated to have a physiological benefit or provide protection against chronic disease. Accordingly, the compounds described for use herein can also be administered as nutraceutical or nutritional formulations, with additives such as nutraceutically or nutritionally acceptable excipients, nutraceutically or nutritionally acceptable carriers, and nutraceutically or nutritionally acceptable vehicles. Such formulations are sometimes called medical foods. Suitable nutraceutically acceptable excipients may include liquid solutions such as a solution comprising one or more vegetable-derived oils, such as sesame oil, and/or one or more animal-derived oils, and/or one or more fish-derived oils.

The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

Injectable preparations, for example, sterile injectable aqueous or oleaginous suspensions, may be formulated according to methods known in the art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents. Alternatively, the compound may also be administered in neat form if suitable.

The compounds for use in the present invention can also be administered in the form of liposomes. As is known in the art, liposomes are generally derived from phospholipids or other lipid substances. Liposomes are formed by mono or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound for use in the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The amount of active ingredient that may be combined with the carrier materials to produce a single dosage form can vary depending upon the patient to which the active ingredient is administered and the particular mode of administration. It will be understood, however, that the specific dose level for any particular patient will depend upon a variety of factors including the activity of the specific compound employed; the age, body weight, body area, body mass index (BMI), general health, sex, and diet of the patient; the time of administration and route of administration used; the rate of excretion; drug combination, if any, used; and the progression and severity of the disease in the patient undergoing therapy. The pharmaceutical unit dosage chosen is usually fabricated and administered to provide a defined final concentration of drug in the blood, cerebrospinal fluid, brain tissues, spinal cord tissues, other tissues, other organs, or other targeted region of the body.

Compounds for use in the present invention may be administered in a single daily dose, or the total daily dosage may be administered in divided dosage of two, three or four times daily.

While the compounds for use in the present invention can be administered as the sole active pharmaceutical agent, they can also be used in combination with one or more other agents used in the treatment or suppression of disorders.

When additional active agents are used in combination with the compounds for use in the present invention, the additional active agents may generally be employed in therapeutic amounts as indicated in the Physicians' Desk Reference (PDR) 53rd Edition (1999), which is incorporated herein by reference, or such therapeutically useful amounts as would be known to one of ordinary skill in the art, or as are determined empirically for each patient.

The compounds for use in the present invention and the other therapeutically active agents can be administered at the recommended maximum clinical dosage or at lower doses. Dosage levels of the active compounds in the compositions for use in the present invention may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

In one embodiment, the purity of the preparation of the compound, such as a tocotrienol quinone preparation, is measured prior to the addition of any pharmaceutical carriers or excipients, or any additional active agents. For example, if alpha-tocotrienol quinone is prepared according to any of the methods described in International Patent Application No. PCT/US2009/062212 or U.S. patent application Ser. No. 12/606,923, the purity of the alpha-tocotrienol quinone is measured on the final product of the method selected, and prior to adding the pharmaceutical carrier(s) or excipient(s) or additional active agent(s). The purity of the desired tocotrienol quinone, or other compound, by weight, can be at least about 20%, at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99%, prior to the addition of any pharmaceutical carriers or excipients, or any additional active agents. These same numerical purity levels can also be used as by mole fraction, or by any other relative measurement (such as weight/volume).

In another embodiment, the purity of the preparation of the compound, such as a tocotrienol quinone preparation, is measured as a fraction of the desired tocotrienol quinone relative to the total amount of tocotrienol quinones and (if present) tocotrienols in the preparation. For example, a composition containing 100 mg of alpha-tocotrienol quinone, 50 mg of beta-tocotrienol quinone, and 50 mg of gamma-tocotrienol hydroquinone would be described as 50% alpha tocotrienol quinone by weight, irrespective of the amounts of other non-tocotrienol or non-tocotrienol quinone compounds present in the preparation. This measurement of purity would be the same whether measured before or after addition of pharmaceutical carriers or excipients, or before or after addition of any non-tocotrienol/non-tocotrienol quinone active agents. The purity of the desired tocotrienol quinone, or other compound, by weight, can be at least about 20%, at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99%. These same numerical purity levels can also be used as by mole fraction, or by any other relative measurement (such as weight/volume).

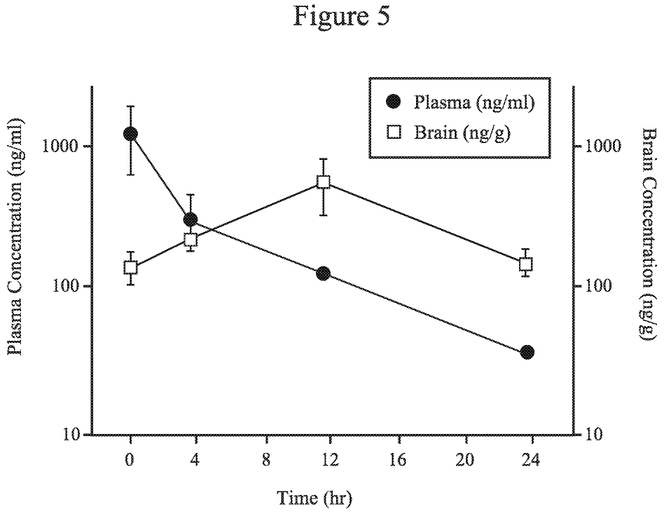

While it is preferable to administer compounds that cross the blood-brain barrier, compounds that do not cross the blood-brain barrier can be delivered to the central nervous system by spinal and epidural administration, or administration to cerebral ventricles. FIG. 5 indicates that alpha-tocotrienol quinone crosses the blood-brain barrier in mice (the data shown is from homogenized brains from C57/BL mice dosed IP at 25 mg/kg).

### Kits

The invention also provides articles of manufacture and kits containing materials useful for treating Leigh Syndrome or Leigh-like Syndrome. The article of manufacture comprises a container with a label. Suitable containers include, for example, bottles, vials, and test tubes. The containers may be formed from a variety of materials such as glass or plastic. The container holds a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone. In one embodiment, the compound is alpha-tocotrienol quinone. In one embodiment, the active agent is alpha-tocotrienol quinone. The label on the container indicates that the composition is used for treating Leigh Syndrome or Leigh-like Syndrome, and may also indicate directions for use in treatment.

The invention also provides kits comprising any one or more of a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone. In some embodiments, the kit of the invention comprises the container described above, which holds a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone. In other embodiments, the kit of the invention comprises the container described above, which holds a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, and a second container comprising a vehicle for the compound or composition, such as one or more vegetable-derived oils, such as sesame oil, and/or one or more animal-derived oils, and/or one or more fish-derived oils. In other embodiments, the kit of the invention comprises the container described above, which holds a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, where the compound or composition has been pre-mixed with a vehicle for the compound or composition, such as one or more vegetable-derived oils, such as sesame oil, and/or one or more animal-derived oils, and/or one or more fish-derived oils. The kits may further include other materials desirable from a commercial and user standpoint, including other vehicles, buffers, diluents, filters, needles, syringes, and package inserts with instructions for performing any of the methods described herein for treatment of Leigh Syndrome or Leigh-like Syndrome.

In other aspects, the kits may be used for any of the methods described herein, including, for example, to treat an individual with Leigh Syndrome or Leigh-like Syndrome.

### EXAMPLES

### Example 1

### Surfeit-1 (SURF1) Cell Line Assay and Initial Screen for Effective Compounds

Alpha-Tocotrienol quinone, its redox-silent version (the bis-Boc protected corresponding hydroquinone), and solvent controls were tested for their ability to rescue cells from SURF-1 fibroblasts of the patient diagnosed with SURF-1 described in Example 2, when the cells were stressed by addition of L-buthionine-(S,R)-sulfoximine (BSO), as described in Jauslin et al., Hum. Mol. Genet. 11(24):3055 (2002), Jauslin et al., FASEB J. 17:1972-4 (2003), and International Patent Application WO 2004/003565. EC₅₀ concentrations of test compound and its redox-silent version were determined and compared. The following compound, 2-((6E,10E)-3-hydroxy-3,7,11,15-tetramethylhexadeca-6,10,14-trienyl)-3,5,6-trimethyl-bis(t-butyloxycarbonyl)benzene-1,4-diol, the bis-Boc protected hydroquinone form of alpha-tocotrienol quinone, was used as the “redox-silent” alpha tocotrienol quinone, or αTTQ-RS.

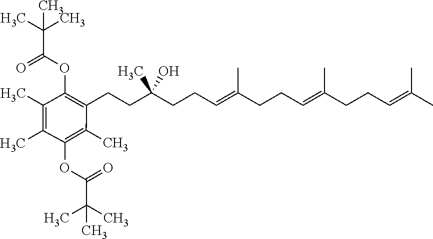 [SMILES: CC(C)=CCC/C(C)=C/CC/C(C)=C/CC[C@@](C)(O)CCc1c(C)c(OC(=O)C(C)(C)C)c(C)c(C)c1OC(=O)C(C)(C)C]

MEM (a medium enriched in amino acids and vitamins, catalog no. 1-31F24-I) and Medium 199 (M199, catalog no. 1-21F22-I) with Earle's Balanced Salts, without phenol red, were purchased from Bioconcept. Fetal Calf Serum was obtained from PAA Laboratories. Basic fibroblast growth factor and epidermal growth factor were purchased from PeproTech. Penicillin-streptomycin-glutamine mix, L-buthionine (S,R)-sulfoximine, and insulin from bovine pancreas were purchased from Sigma. Calcein AM was purchased from Molecular Probes. Cell culture medium was made by combining 125 mL M199 EBS, 50 ml Fetal Calf Serum, 100 U/mL penicillin, 100 μg/ml streptomycin, 2 mM glutamine, 10 μg/mL insulin, 10 ng/mL EGF, and 10 ng/mL bFGF. MEM EBS was added to make the volume up to 500 mL. A 10 mM BSO solution was prepared by dissolving 444 mg BSO in 200 mL of medium with subsequent filter-sterilization. During the course of the experiments, this solution was stored at +4° C. The cells were obtained from the SURF-1 patient and grown in 10 cm tissue culture plates. Every third day, they were split at a 1:3 ratio.

The test samples were supplied in 1.5 mL glass vials. The compounds were diluted with DMSO, ethanol or PBS to result in a 5 mM stock solution. Once dissolved, they were stored at −20° C.

Test samples were screened according to the following protocol: A culture with SURF-1 fibroblasts was started from a 1 mL vial with approximately 500,000 cells stored in liquid nitrogen. Cells were propagated in 10 cm cell culture dishes by splitting every third day in a ratio of 1:3 until nine plates were available. Once confluent, fibroblasts were harvested. For 54 micro titer plates (96 well-MTP) a total of 14.3 million cells (passage eight) were re-suspended in 480 mL medium, corresponding to 100 μL medium with 3,000 cells/well. The remaining cells were distributed in 10 cm cell culture plates (500,000 cells/plate) for propagation. The plates were incubated overnight at 37° C. in an atmosphere with 95% humidity and 5% CO₂ to allow attachment of the cells to the culture plate.

MTP medium (243 μL) was added to a well of the microtiter plate. The test compounds were unfrozen, and 7.5 μL of a 5 mM stock solution was dissolved in the well containing 243 μL medium, resulting in a 150 μM master solution. Serial dilutions from the master solution were made. The period between the single dilution steps was kept as short as possible (generally less than 1 second).

Plates were kept overnight in the cell culture incubator. The next day, 10 μL of a 10 mM BSO solution were added to the wells, resulting in a 1 mM final BSO concentration. Forty-eight hours later, three plates were examined under a phase-contrast microscope to verify that the cells in the 0% control (wells E1-H1) were clearly dead. The medium from all plates was discarded, and the remaining liquid was removed by gently tapping the plate inversed onto a paper towel.

100 μL of PBS containing 1.2 μM Calcein AM were then added to each well. The plates were incubated for 50-70 minutes at room temperature. After that time the PBS was discarded, the plate gently tapped on a paper towel and fluorescence (excitation/emission wavelengths of 485 nm and 525 nm, respectively) was read on a Gemini fluorescence reader. Data was imported into Microsoft Excel (EXCEL is a registered trademark of Microsoft Corporation for a spreadsheet program) and used to calculate the EC₅₀ concentration for each compound.

The compounds were tested three times, i.e., the experiment was performed three times, the passage number of the cells increasing by one with every repetition.

The solvents (DMSO, ethanol, PBS) neither had a detrimental effect on the viability of non-BSO treated cells nor did they have a beneficial influence on BSO-treated fibroblasts even at the highest concentration tested (1%). The compounds showed no auto-fluorescence. The viability of non-BSO treated fibroblasts was set as 100%, and the viability of the BSO— and compound-treated cells was calculated as relative to this value.

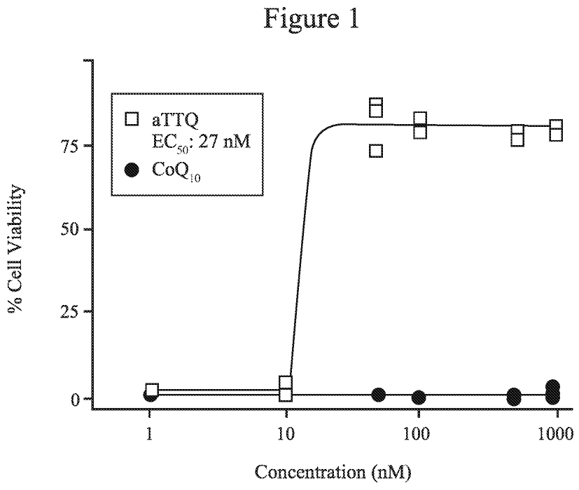

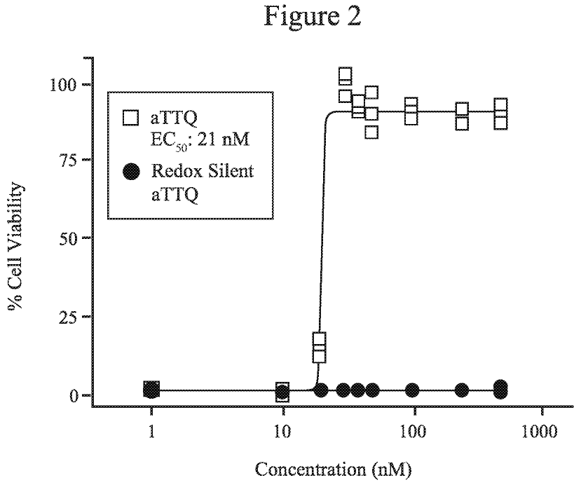

The results of the cell viability assay for the SURF1-mutant cells in the presence of alpha-tocotrienol quinone (αTTQ) and redox-silent alpha-tocotrienol quinone (αTTQ-RS) are shown in FIG. 2. Alpha-tocotrienol quinone protects the cells with an ED₅₀ of 21 nM (comparable to the ED₅₀ of 27 nM determined in FIG. 1), while redox-silent alpha-tocotrienol quinone is ineffective at maintaining cell viability of the fibroblasts cells from the patient of Example 2.

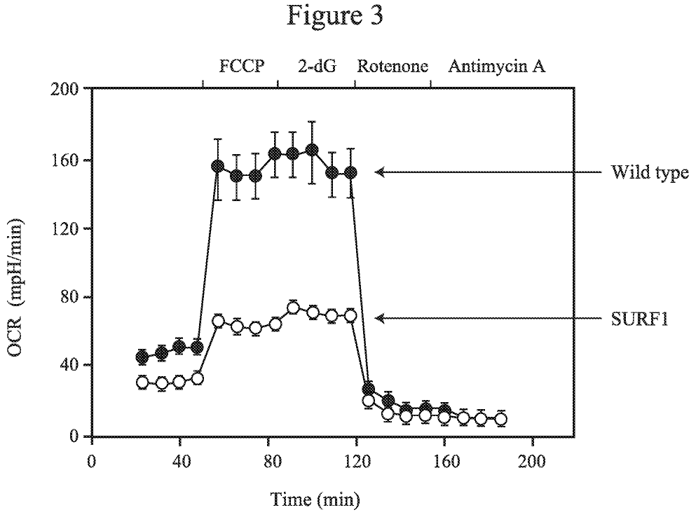

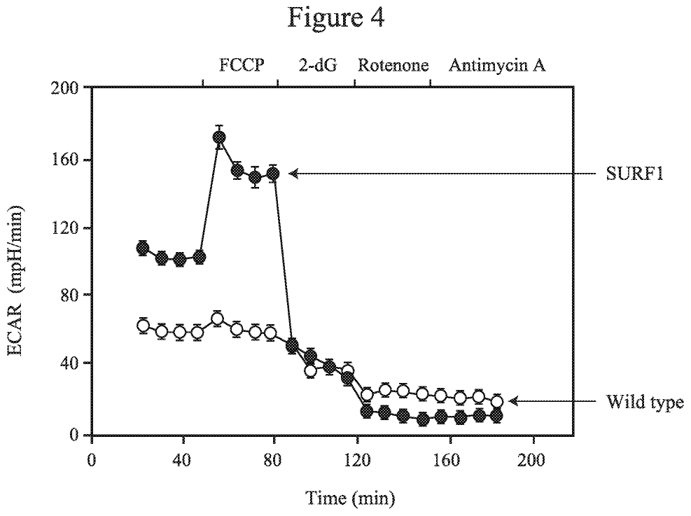

Cells from the subject treated in Example 2 were characterized using various compounds. FIG. 3 shows the oxygen consumption rate (OCR) of cells in the presence of carbonylcyanide p-trifluoromethoxyphenylhydrazone (FCCP), 2-deoxyglucose (2-dG), rotenone, and Antimycin A, while FIG. 4 shows the Extracellular Acidification Rate (ECAR) of cells from the subject treated in Example 2, in the presence of carbonylcyanide p-trifluoromethoxyphenylhydrazone (FCCP), 2-deoxyglucose (2-dG), rotenone, and Antimycin A. The agents are added sequentially, and all four agents are present in the medium at the end of the experiment.

### Example 2

### Treatment of a Leigh Syndrome Patient Diagnosed with Surfeit-1 (SURF-1) Mutation

A four-year-old female patient with Leigh Syndrome was treated with alpha-tocotrienol quinone. Informed consent was obtained from the child's parents in accordance with federal regulations and institutional protocol. Two mutations were identified in the SURF-1 gene of the patient.

The patient's weight was approximately 10 kg. Alpha-tocotrienol quinone was administered to the patient via gastrointestinal feeding tube; the drug was mixed with sesame oil for administration. The following dosing of alpha-tocotrienol quinone was used:

### Days 0-5: 0 mg

Days 5, 7-20: 32 mg (3.2 mg/kg)
Days 20, 22-50: 80 mg (8 mg/kg)
Days 50, 52-80: 120 mg (12 mg/kg)
Days 80, 82 and continuing: 200 mg (20 mg/kg)

The day after each increase in dosage, no dose was administered, and laboratory tests were performed to evaluate the effect of the increased dosage on clinical markers.

Full pharmacokinetic sampling (post dose, 3 h, 6 h, 10 h, 24 h, 36 h, 48 h, and 72 h) was done after administration of the initial dose. 0.5 mL of blood were taken at each time point, for a total of four mL of blood drawn at 72 h. Bio-analysis and pharmacokinetic calculations were conducted.

The minimal effective dose of alpha-tocotrienol quinone was determined. The concentration 24 hours after dosing (C₂₄ₕ) was expected to be 220.5 ng/ml (0.5 μM), while the minimal effective dose was expected to be around 150 mg. Thus, the daily dosage was escalated stepwise. After each single dose escalation phase, a new dose was then tested if pharmacokinetic results indicate a change in dose was required to meet the anticipated minimal effective dose. Pharmacokinetic sampling was done pre-dose, at Tₘₐₓ and 24 h, and 48 h or even 72 h if considered as necessary from the first pharmacokinetic results.

While being treated with alpha tocotrienol quinone, the patient's medical team continued to assess blood levels of alpha tocotrienol quinone to determine the correct dose. Other lab specimens were obtained as explained below. In addition, the medical team monitored the patient for any signs of improvement or signs of worsening of the disease.

The following schedule was used for the first two weeks of treatment:

WEEK 1 (Day 1-Day 5): no alpha-tocotrienol quinone was administered; baseline data were collected

WEEK 2 (Day 6 to Day 10): administration of 32 mg alpha-tocotrienol quinone on Day 6 (in two doses of 16 mg each); no dosing on Day 7; administration of 32 mg alpha-tocotrienol quinone on Days 8-10.

The first day of administration of the investigational drug alpha tocotrienol quinone was designated as Day 6. No alpha tocotrienol quinone was administered on Day 7 (see Table 3). During these first 48 hours (Day 6 and Day 7), lab specimens were collected and processed that allowed the patient's medical management team to evaluate how the patient's body processed the drug. On Day 8, dosing continued on a daily basis.

TABLE 3
Dosing Schedule for initial five days

| Day 6 | Day 7 | Day 8 | Day 9 | Day 10 |
|---|---|---|---|---|
| 1^{st} dose | No dose | 2^{nd} dose | 3^{rd} dose | 4^{th} dose |
| 1.6 mg/kg | No dose | 1.6 mg/kg | 1.6 mg/kg | 1.6 mg/kg |

The blood specimens collected in the first 48 hours were designated “pharmacokinetic samples” and were collected according to the schedule on Table 4.

TABLE 4

| Before the | | | | | | | |
|---|---|---|---|---|---|---|---|
| 1^{st} dose of | | | | | | | |
| alpha | Pharmacokinetic Blood Testing Schedule | | | | | | |
| tocotrienol | After Hour 0 and up to Hour 48 | | | | | | |
| quinone | (Day 6 and Day 7) | | | | | | |
| 0 hr | 1 hr | 3 hrs | 6 hrs | 10 hrs | 24 hrs | 36 hrs | 48 hrs |
| 1.5 | 1.5 | 1.5 | 1.5 | 1.5 | 1.5 | 1.5 | 1.5 |
| mL | mL | mL | mL | mL | mL | mL | mL |

The total amount of blood taken for determining the level of alpha tocotrienol quinone on Day 6 was a total of 15 mL.

Additional Testing Days 6-10:

Table 5 summarizes the additional testing performed on Days 6-10. In addition to the “pharmacokinetic samples” that were collected, a separate blood specimen was obtained to analyze the patient's metabolic profile on Day 6. For this specimen, a total amount of 1.5 mL was obtained prior to the administration of the first dose of alpha tocotrienol quinone on Day 6. Urine specimens were used to analyze the patient's metabolic profile in the urine. The quantity of urine needed for each sample was approximately 5 mL. On Day 6 through Day 10, after a period of 3 hours (±30 minutes) after drug administration, an electrocardiogram was obtained. ECGs after Day 10 were obtained as directed by the medical management team.

TABLE 5
BLOOD, URINE METABOLOMIC AND ECG SCHEDULE

| Description of Test | Day 6 | Day 7 | Day 8 | Day 9 | Day 10 |
|---|---|---|---|---|---|
| Blood Metabolomics (1.5 mL) | √ | — | — | — | — |
| Urine Metabolomics (5 mL) | √ | — | — | — | — |
| ECG | √ | √ | √ | √ | √ |
| Complete Metabolic Profile* | √ | — | — | — | √ |
| CBC with differential | √ | — | — | — | √ |
| PT/PTT/INR | √ | — | — | — | √ |

*Complete Metabolic Profile indicates Blood Chemistry values that include electrolytes, liver function tests, and kidney function testing

After the First Week of Dosing (after Day 10):

Further dosing, monitoring and blood tests were performed as directed by the treating physicians.

End of Repeat Dosing Visit:

The patient returned for an outpatient visit that included measurements of clinical laboratory assessment (hematology, chemistry, and urinalysis), sampling for blood- and urine-derived analytes, plasma sample for drug pre-dose concentration determination, physical examination and vital signs, safety ECG, and body weight. An appointment for a magnetic resonance spectroscopy evaluation was made and the patient was discharged.

Final Study Visit:

The patient returned for a review of safety and efficacy evaluation, including CNS lactate/pyruvate ratios as determined by non-invasive means (MRI/MRS) and ECHO. The following efficacy parameters were evaluated: PK parameters; magnetic resonance spectroscopy of central nervous system; echocardiography and electrocardiogram; and plasma and urine analytes.

Lactic acid measurements were as follows:

In cerebrospinal fluid: about 2 years pre-treatment, the patient's lactate levels were measured at 46.5 mg/dL. After treatment, CSF lactate levels were measured at 24.0 mg/dL, a 48.4% reduction.

In brain: the decrease in lactate from two days prior to treatment to approximately 98 days after starting treatment was approximately 20-30%.

In plasma: approximately 4 months prior to treatment, the patient's lactate levels were measured at 3.6 mM. After treatment, plasma lactate levels were measured at 1.1 mM, a 69.4% reduction, and within the normal plasma lactate range of 1.0 to 1.4 mM.

Magnetic Resonance Imaging:

Magnetic resonance imaging of the patient was performed to assess the effect of treatment. Prior to treatment, MRI revealed new patchy areas of T2 hyperintensity with restricted diffusion and contrast enhancement, involving the brainstem, cerebral and cerebellar peduncles and the deep cerebellum. These findings are consistent with Leigh Syndrome encephalopathies.

MRI after treatment with alpha-tocotrienol quinone showed (in comparison to the pre-treatment MRI) interval improvement of previously noted T2 hyperintensities involving the midbrain, pons, medulla and cerebellum. An overall decrease in the amount of lesions was also observed.

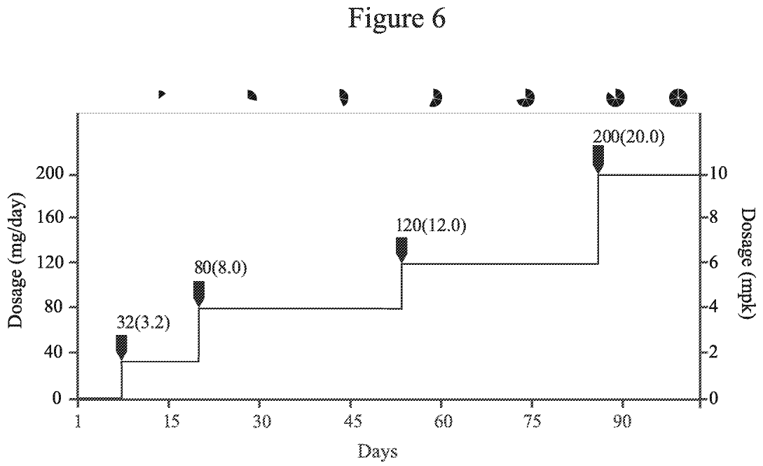

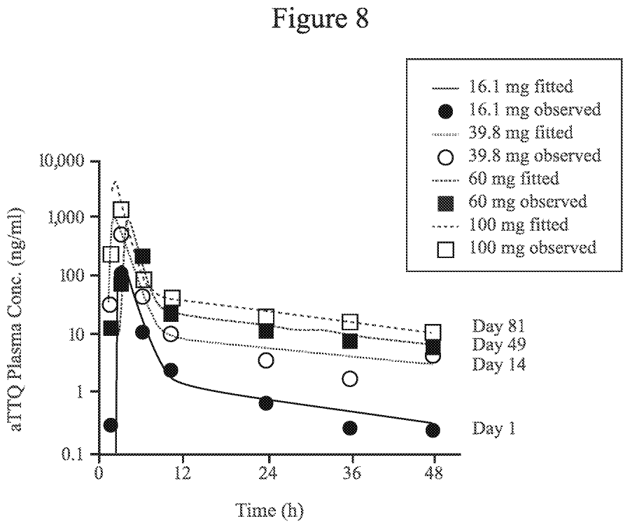

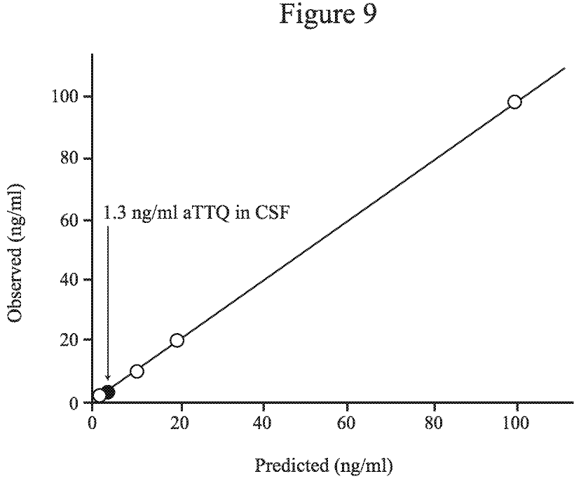

The concentration of alpha tocotrienol quinone was monitored in the patient. FIG. 6 is a graph of the dosage administered to the subject, versus day of treatment, while FIG. 8 shows the plasma concentration of alpha tocotrienol quinone (ng/ml) and FIG. 9 shows the cerebrospinal fluid (CSF) concentration of alpha tocotrienol quinone (ng/ml) in the subject. Alpha tocotrienol quinone was present at 1.3 ng/ml in CSF.

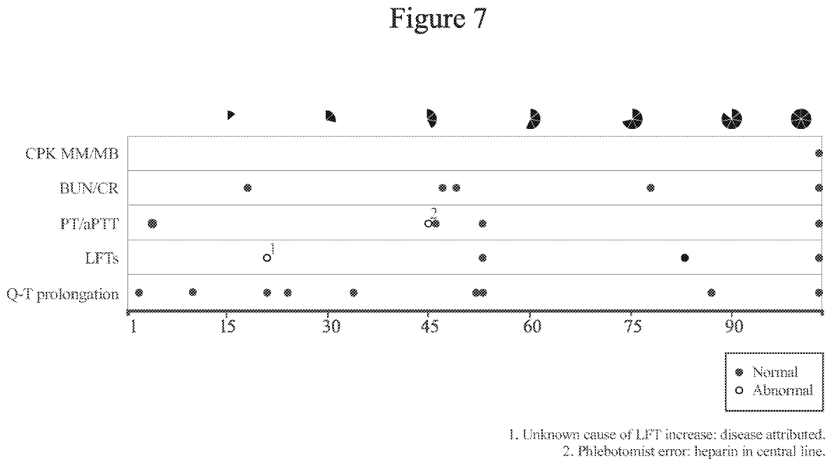

Close monitoring of the patient during the study was performed, to detect any adverse events. In addition, the investigator had authority to stop the study if the safety of the subject was at risk. No adverse events were observed; see FIG. 7 for a diagram of events observed in the patient.

### Example 3

### Treatment of a Leigh Syndrome Patient

A 4-year-old male patient with Leigh Syndrome was treated with alpha-tocotrienol quinone. Informed consent was obtained from the child's parents in accordance with federal regulations and institutional protocol. A mutation was identified in the SURF-1 gene of the patient.

At the start of treatment, the patient was unable to control his extremities. After 22 days of treatment, the boy was able to move his arm in the “high-five” gesture. Gastrointestinal function was greatly improved, and sleep was improved. The patient also gained weight. After 379 days of treatment, the patient had gained 15 pounds, was able to sit upright, rode on a horse with assistance, and started kindergarten.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1.-19. (canceled)

20. A composition comprising: alpha-tocotrienol quinone, alpha-tocotrienol hydroquinone, or a mixture thereof; and sesame oil.

21. The composition of claim 20, wherein the composition is a pharmaceutical preparation, and wherein the alpha-tocotrienol quinone, alpha-tocotrienol hydroquinone, or mixture thereof, is present in a therapeutically effective amount.

22. The composition of claim 21, wherein the alpha-tocotrienol quinone comprises at least about 80% by weight of the tocotrienols and tocotrienol quinones present in the preparation.

23. The composition of claim 21, wherein the alpha-tocotrienol quinone comprises at least about 90% by weight of the tocotrienols and tocotrienol quinones present in the preparation.

24. The composition of claim 21, wherein the alpha-tocotrienol quinone comprises at least about 90% by weight of the material present in the preparation, excluding the weight of pharmaceutical carriers and excipients.

25. The composition of claim 20, wherein the alpha-tocotrienol quinone, alpha-tocotrienol hydroquinone, or mixture thereof is present as the naturally occurring configuration.

26. The composition of claim 21, wherein the alpha-tocotrienol quinone, alpha-tocotrienol hydroquinone, or mixture thereof is present as the naturally occurring configuration.

27. The composition of claim 23, wherein the alpha-tocotrienol quinone, alpha-tocotrienol hydroquinone, or mixture thereof is present as the naturally occurring configuration.

28. The composition of claim 21, wherein the preparation is formulated for administration of a unit dosage form of between about 50 mg to 500 mg of alpha-tocotrienol quinone.

29. The composition of claim 23, wherein the preparation is formulated for administration of a unit dosage form of between about 50 mg to 500 mg of alpha-tocotrienol quinone.

30. The composition of claim 27, wherein the preparation is formulated for administration of a unit dosage form of between about 50 mg to 500 mg of alpha-tocotrienol quinone.

31. The composition of claim 21, wherein the preparation is formulated for administration of alpha-tocotrienol quinone, alpha-tocotrienol hydroquinone, or a mixture thereof at about 1.0 mg/kg to about 30 mg/kg body weight.

32. The composition of claim 23, wherein the preparation is formulated for administration of alpha-tocotrienol quinone, alpha-tocotrienol hydroquinone, or a mixture thereof at about 1.0 mg/kg to about 30 mg/kg body weight.

33. The composition of claim 27, wherein the preparation is formulated for administration of alpha-tocotrienol quinone, alpha-tocotrienol hydroquinone, or a mixture thereof at about 1.0 mg/kg to about 30 mg/kg body weight.

34. The composition of claim 21, wherein the composition is formulated for enteral administration.

35. The composition of claim 21, wherein the composition is formulated for oral administration.

36. The composition of claim 21, wherein the composition is formulated for administration via feeding tube, feeding syringe, or gastrostomy.

37. The composition of claim 20, wherein the composition is a nutraceutical or nutritional formulation.

38. The composition of claim 37, wherein the composition is a medical food.
